(19) United States
(12) Patent Application Publication
(10) Pub. No.: US 2010/0040630 A1
(43) Pub. Date: Feb. 18, 2010

(54) METHODS AND COMPOSITIONS FOR TREATING BONE LOSS

(76) Inventors: Aake Elden, Jar (NO); Glenn Haugeberg, Kristiansand S. (NO)

Correspondence Address: MCCARTER & ENGLISH, LLP BOSTON, 265 Franklin Street, Boston, MA, 02110, US

(21) Appl. No.: 12/410,321

(22) Filed: Mar. 24, 2009

Related U.S. Application Data
(60) Provisional application No. 61/148,313, filed on Jan. 29, 2009.
(60) Provisional application No. 61/039,028, filed on Mar. 24, 2008.

(51) Int. Cl. A61K 39/395; A61P 19/08

(52) U.S. Cl. 424/142.1; 424/158.1

(57) ABSTRACT

The invention provides methods and compositions for treating, e.g., reducing, bone loss, e.g., cortical bone loss, comprising administering a TNFα inhibitor, such as a human TNFα antibody, or antigen-binding portion thereof.

### RELATED APPLICATIONS

This application claims the benefit of priority to U.S. Provisional Appln. No. 61/039,028, filed on Mar. 24, 2008, and U.S. Provisional Appln. No. 61/148,313, filed on Jan. 29, 2009. The contents of all the above-mentioned priority applications are hereby incorporated by reference in their entirety.

### BACKGROUND OF THE INVENTION

Bone loss is characterized by structural deterioration of bone tissue, which can lead to bone fragility and an increased susceptibility to fractures. Bone loss is associated with a number of diseases, including osteoporosis, osteoarthritis, and rheumatoid arthritis.

In rheumatoid arthritis (RA), for example, bone damage on radiographs presents not only as erosions but also as periarticular osteoporosis.[1] There are data for the importance of suppressing inflammation to avoid bone damage in RA. Through results of several randomised controlled clinical trials, potent anti-inflammatory treatments have been shown to reduce the progression of joint erosions.[4-6] Inflammatory activation of the osteoclast is involved in both features,[2,3] and the suppression of osteoclast activity has been shown to reduce the progression of erosions using bisphosphonate zoledronic acid [3].

A few studies have suggested that anti-TNF therapy may have the ability to prevent general bone loss. For example, the anti-inflammatory effect of anti-tumour necrosis factor (anti-TNF) therapy has been shown to significantly reduce the progression of radiographic joint damage in RA patients. [4-6] There is also evidence that anti-inflammatory treatment reduces generalised osteoporosis. [7-9]

Quantitative hand bone measures have been recommended for their sensitivity in assessing inflammatory bone involvement in early RA.[10] However, only a few studies have examined the effect of anti-inflammatory treatment (including anti-TNF therapy) on hand bone loss in RA. [9,11,12] In one study employing quantitative ultrasound (QUS), use of anti-TNF therapy had a positive effect on periarticular bone.[11] Only one randomised controlled trial has been conducted, however, where the anti-inflammatory effects of prednisolone (7.5 mg daily) compared with placebo were shown to significantly reduce not only the rate of radiographic joint damage, but also the rate of hand bone loss.[12] In addition to its anti-inflammatory effect, however, prednisolone is also known to cause osteoporosis.[12] As such, there remains a need for therapeutic agents for treating bone loss, especially hand bone loss.

### SUMMARY OF THE INVENTION

The invention provides a method for treating bone loss, e.g., reducing and/or preventing bone loss, in subject comprising administering a TNFα inhibitor, e.g., an antibody, or antigen-binding portion thereof, to the subject. In one embodiment bone loss in the hand of the subject is treated. In one embodiment, cortical hand bone loss is treated.

In one embodiment, the subject has a disorder associated with bone loss. In one embodiment, the subject has osteoporosis. In one embodiment, the subject has osteoarthritis. In yet another embodiment, the subject has rheumatoid arthritis (RA). In one embodiment, the subject has osteoporosis and RA.

In one embodiment of the invention, the TNFα inhibitor is administered in combination with an additional agent. In one embodiment, TNFα inhibitor is administered in combination with methotrexate. In another embodiment, the TNFα inhibitor is administered in combination with an antiresorptive agent, e.g., alendronate, alendronate plus vitamin D3, ibandronate, risedronate, risedronate with calcium, zoledronic acid, calcitonin, estrogen, and, raloxifene. In yet another embodiment, the TNFα inhibitor is administered in combination with a bone forming agent, such as a parathyroid hormone, e.g., teriparatide.

In one embodiment, the subject who is administered a TNFα inhibitor for the treatment of bone loss, may be selected for having and/or being at risk of having bone loss. In one embodiment, the invention provides a method for treating hand bone loss in a subject, comprising selecting a subject who has hand bond loss or is at risk of having hand bone loss and administering a TNFα inhibitor to the subject, such that hand bone loss is treated. In another embodiment, method of the invention is performed on a subject who was previously selected as having or at risk of having bone loss.

The invention also provides methods for predicting bone loss, including, but not limited to, hand bone loss. The invention provides indices that may be used to predict bone loss, e.g., hand bone loss, in a subject. For example, the subject's age and/or CRP level may be used to predict hand bone loss in a subject.

In one embodiment of the invention, the TNFα inhibitor is a TNFα antibody, or antigen-binding portion thereof. In one embodiment, the TNFα antibody, or antigen-binding portion thereof, is a human TNFα antibody, or antigen-binding portion thereof. In another embodiment the TNFα antibody, or antigen-binding portion thereof, is inliximab or golimumab. In one embodiment, the human TNFα antibody, or an antigen-binding portion thereof, dissociates from human TNFα with a Kd of 1×10⁻⁸ M or less and a Koff rate constant of 1×10⁻³ s⁻¹ or less, both determined by surface plasmon resonance, and neutralizes human TNFα cytotoxicity in a standard in vitro L929 assay with an IC50 of 1×10⁻⁷ M or less. In another embodiment, the human TNFα antibody, or an antigen-binding portion thereof, has the following characteristics: dissociates from human TNFα with a K_{off} rate constant of 1×10⁻³ s⁻¹ or less, as determined by surface plasmon resonance; has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 8 and/or 9; and has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12. In one embodiment, the human TNFα antibody, or an antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprising a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. In one embodiment, the human TNFα antibody, or an antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2. In one embodiment, the human TNFα antibody, or an antigen-binding portion thereof, is adalimumab. In another embodiment, the human TNFα antibody, or an antigen-binding portion thereof, is golimumab.

### BRIEF DESCRIPTION OF THE DRAWINGS

FIG. 1 is a flow-chart of the examined patients with early rheumatoid arthritis in the present analysis. Numbers of missing X-rays compared with the original Study J are provided in parentheses. MTX=methotrexate; DXR=digital X-ray radiogrammetry; MCI=metacarpal cortical index; BMD=bone mass density.

FIG. 2 shows changes in DXR-MCI (percentage) and modified Sharp score (units) over time in the three treatment groups of Study J (A=median values, B=mean values). Mod. Sharp score=modified total Sharp score; MTX=methotrexate; DXR=digital X-ray radiogrammetry; MCI=metacarpal cortical index.

FIG. 3 is a cumulative probability plot—Changes in DXR-MCI and radiographic scores at 104 weeks in Study J. Mod. Sharp score=modified total Sharp score; MTX=methotrexate; DXR=digital X-ray radiogrammetry; MCI=metacarpal cortical index.

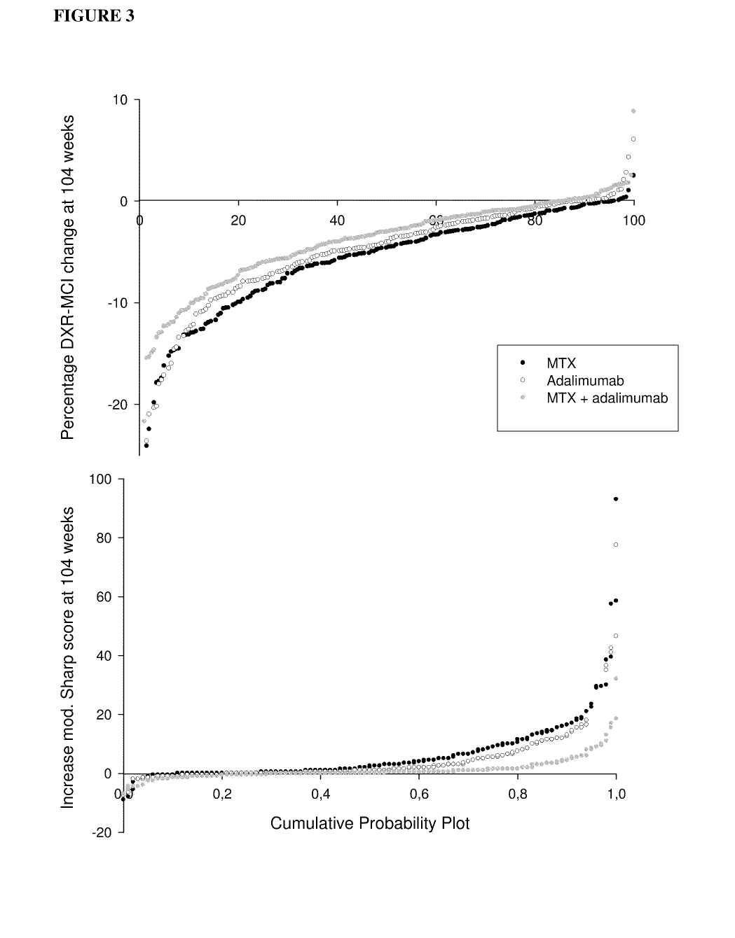

### DETAILED DESCRIPTION OF THE INVENTION

### I. Definitions

The term “human TNFβ” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFβ is intended to include recombinant human TNFα (rhTNFα), which can be prepared by standard recombinant expression methods or purchased commercially (R & D Systems, Catalog No. 210-TA, Minneapolis, Minn.). TNFα is also referred to herein as TNF.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356, each of which is incorporated by reference herein. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (CIMZIA®, a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406,476, incorporated by reference herein). In another embodiment, the TNFα inhibitor is a recombinant TNF binding protein (r-TBP-I) (Serono).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The antibodies of the invention are described in further detail in U.S. Pat. Nos. 6,090,382; 6,258,562; and 6,509,015, each of which is incorporated herein by reference in its entirety.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)2, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH or VL domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Such chimeric, humanized, human, and dual specific antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539.

An “isolated antibody”, as used herein, is intended to refer to an antibody that is substantially free of other antibodies having different antigenic specificities (e.g., an isolated antibody that specifically binds hTNFα is substantially free of antibodies that specifically bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jönsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

The term “Koff”, as used herein, is intended to refer to the off rate constant for dissociation of an antibody from the antibody/antigen complex.

The term “Kd”, as used herein, is intended to refer to the dissociation constant of a particular antibody-antigen interaction.

The term “IC50” as used herein, is intended to refer to the concentration of the inhibitor required to inhibit the biological endpoint of interest, e.g., neutralize cytotoxicity activity.

The term “dose,” as used herein, refers to an amount of TNFα inhibitor which is administered to a subject.

The term “dosing”, as used herein, refers to the administration of a substance (e.g., an anti-TNFα antibody) to achieve a therapeutic objective (e.g., treatment of bone loss).

A “dosing regimen” describes a treatment schedule for a TNFα inhibitor, e.g., a treatment schedule over a prolonged period of time and/or throughout the course of treatment, e.g. administering a first dose of a TNFα inhibitor at week 0 followed by a second dose of a TNFα inhibitor on a biweekly dosing regimen. In one embodiment, the dosing regimen includes administering a TNFα inhibitor, e.g, a human TNFα antibody, or antigen binding portion thereof, once a month or once every four weeks.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g, throughout the course of treatment. The biweekly dosing regimen is not intended to include a weekly dosing regimen. The substance may, for example, be administered every 9-19 days, more preferably, every 11-17 days, even more preferably, every 13-15 days, and most preferably, every 14 days. In one embodiment, the biweekly dosing regimen is initiated in a subject at week 0 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen wherein doses of a TNFα inhibitor are administered to a subject every other week beginning at week 0. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-TNFα antibody and another drug. The other drug(s) may be administered concomitant with, prior to, or following the administration of an anti-TNFα antibody.

The term “treatment,” as used within the context of the present invention, is meant to include therapeutic treatment, as well as prophylactic or suppressive measures, for the treatment of bone loss, e.g., hand bone loss, e.g., cortical hand bone loss. For example, the term treatment may include administration of a TNFα inhibitor prior to or following the onset of bone loss, e.g., hand bone loss, thereby preventing or removing signs of the disease or disorder. As another example, administration of a TNFα inhibitor after clinical manifestation of bone loss to combat the symptoms and/or complications and disorders associated with bone loss comprises “treatment” of the disease. Further, administration of the agent after onset and after clinical symptoms and/or complications have developed where administration affects clinical parameters of the disease or disorder and perhaps amelioration of the disease, comprises “treatment” of bone loss. In one embodiment, treatment of bone loss in a subject comprises reducing signs and symptoms.

Those “in need of treatment” include mammals, such as humans, already having bone loss, including those in which the disease or disorder is to be prevented.

The invention generally provides improved uses and compositions for treating bone loss, e.g., hand bone loss, e.g., cortical hand bone loss, with a TNFα inhibitor, e.g., a human TNFα antibody, or an antigen-binding portion thereof. Compositions and articles of manufacture, including kits, relating to the methods and uses for treating bone loss are also contemplated as part of the invention. Various aspects of the invention are described in further detail herein.

### II. Uses and Compositions for Treating Bone Loss

The instant invention provides a means of treating bone loss, including hand bone loss, by administering a TNFα inhibitor, e.g., a TNFα antibody, or antigen-binding portion thereof, to a subject in need thereof. In one embodiment, the method of the invention may be used to treat a subject having bone loss associated with another disorder, including, for example, rheumatoid arthritis, osteoarthritis, and/or osteoporosis. Subjects who may benefit from the methods of the invention include those subjects who have been diagnosed with bone loss (or a disorder associated with bone loss), as well as subjects identified as being at risk for bone loss (including subjects diagnosed with a disorder associated with bone loss). In one embodiment, the methods of the invention are useful for the treatment of bone loss of the hand.

In one embodiment, a TNFα inhibitor, e.g., a TNFα antibody, or antigen-binding portion thereof, is administered to a subject having bone loss (or a disorder associated with bone loss), such that the progression of the bone loss is arrested, or slowed relative to bone loss without treatment. Thus, the methods of the invention may be used to reduce bone loss in a subject, as well as to prevent further bone loss.

One aspect of the invention relates to the unexpected finding that TNFα inhibitors, e.g., a human TNFα antibody, or antigen-binding portion thereof, may be used to treat hand bone loss. Prior to this finding, a study using the chimeric TNFα antibody infliximab showed that even if bone loss in the hip and spine of treated subjects was arrested, hand bone loss was not halted.[9] Thus, in one embodiment, the methods and compositions of the invention may be used to treat hand bone loss, including hand bone loss associated with RA, osteoarthritis, and osteoporosis. The methods and compositions of the invention may be used to treat hand bone loss in a subject who has or may develop hand bone loss.

In one embodiment, the methods of the invention are useful for the treatment of cortical bone loss. Cortical bone, or compact bone, in contrast to trabecular or cancellous bone, is dense and forms the surface of bones, contributing 80% of the weight of a human skeleton. It is extremely hard, formed of multiple stacked layers with few gaps. Its main function is to support the body, protect organs, provide levers for movement, and (shared with cancellous bone) store minerals. As described herein, one discovery of the examples provided below is that TNFα inhibitors may be used to treat cortical bone loss. In one embodiment, the methods of the invention may be used to treat cortical bone loss of the hand.

The treatment of bone loss, e.g., cortical bone loss or hand bone loss, e.g., cortical hand bone loss, may be assessed using means known in the art, including, but not limited to, digital X-ray radiogrammetry (DXR) (Sectra, Linkoping, Sweden). DXR measures hand bone mineral density (BMD) and metacarpal cortical index (MCI) on the same digitised hand for assessment of radiographic joint damage. DXR is a computer version of the traditional radiogrammetry technique. On hand radiographs, the computer automatically recognizes regions of interest (ROI) around the narrowest part of the second, third, and fourth metacarpal bone and measures cortical thickness, bone width, and porosity 118 times per cm. DXR-BMD is defined as: c X VPAcomb X (1-p), where c is a constant (determined by the result that DXR-BMD, on average, is equal to the mid-distal forearm region of the Hologic QDR-2000 device); VPA is volume per area; and p is porosity. DXR-MCI is defined as the combined cortical thickness divided by the outer cortical diameter and is a relative bone measure independent of bone size, bone length, and image capture setting. Other examples of means by which bone loss can be determined are described in Haugeberg (2008) Best Pract Res Clin Rheumatol 22(6): 1127-39.

In one embodiment, the invention provides a method of improving the DXR-MCI and/or DXR-BMD score of a subject having bone loss comprising administering a TNFα inhibitor, such as a TNFα antibody, or antigen-binding portion thereof, to the subject in need thereof. In one embodiment, an improvement in the DXR-MCI and/or DXR-BMD score of a subject is the maintenance of the DXR-MCI and/or DXR-BMD score of the subject prior to treatment with the TNFα inhibitor. Such maintenance of a DXR-MCI and/or DXR-BMD score indicates that bone loss is not progressing. Alternatively, in one embodiment, improvement in the DXR-MCI and/or DXR-BMD score of the subject being treated for bone loss may be measured by a decreased rate of loss of the DXR-MCI and/or DXR-BMD score. Improvement in the DXR-MCI and/or DXR-BMD score of the subject may be measured relative to the initial baseline score determined prior to treatment. For example, a subject may have a decrease in a DXR-MCI score of 1.4 or less (e.g., −1.4, −1.3, −1.2, −1.1., −1.0, −0.9, −0.8,−0.7, −0.6, −0.5, −0.4, −0.3, −0.2, −0.1, or 0.0 relative to a baseline score) following about 26 weeks of treatment or about 13 treatments of the TNFα inhibitor. In another example, a decrease of less than 0.44 (e.g., −0.43, −0.42, −0.41, −0.40, −0.39, −0.38, −0.37, −0.36, −0.35, −0.34, −0.33, −0.32, −0.31, −0.30, −0.29, −0.28, −0.27, −0.26, −0.25, −0.24, −0.23, −0.22, −0.21, −0.20, −0.19, −0.18, −0.17, −0.16, −0.15, −0.14, −0.13, −0.12, −0.11, −0.10, −0.09, −0.08, −0.07, −0.06, −0.05, −0.04, −0.03, −0.02, −0.01) in the DXR-MCI score of a subject relative to a baseline score indicates treatment of bone loss in a subject.

In one embodiment, the invention provides a method of treating bone loss in a subject comprising administering a human TNFα antibody, or antigen-binding portion thereof, e.g., a human TNFα antibody, or antigen-binding portion thereof, to the subject at week 0 on a biweekly dosing regimen. In one embodiment, a human TNFα antibody, or antigen-binding portion thereof, is subcutaneously administered to a subject having bone loss (or at risk of having bone loss) according to a biweekly dosing regimen. Alternatively, a human TNFα antibody, or antigen-binding portion thereof, is administered to a subject having bone loss (or at risk of having bone loss) according to a monthly dosing regimen, or a dosing regimen whereby the antibody, or antigen-binding portion thereof, is administered once every four weeks.

In one embodiment, bone loss is treated by administering a human TNFα antibody, or antigen-binding portion thereof, on biweekly dosing regimen for at least about 2 weeks, at least about 6 weeks, at least about 12 weeks, at least about 16 weeks, at least about 18 weeks, at least about 20 weeks, at least about 22 weeks, at least about 24 weeks, at least about 30 weeks, at least about 36 weeks, at least about 52 weeks at least about 72 weeks, at least about 96 weeks, at least about 104 weeks, etc. Ranges of values between any of the above recited values are also intended to be included in the scope of the invention, e.g, 23 weeks, 60 week, 64 weeks, etc.

In one embodiment, the TNFα inhibitor, e.g, antibody, or an antigen-binding portion thereof, may also be administered to a subject for the treatment of bone loss for a period defined in months, e.g., 3 months, 6 months, 12 months, 18 months, 24 months, 30 months, 36 months, 42 months, 48 months, 54 months, 60 months, etc. Ranges of values between any of the above recited values are also intended to be included in the scope of the invention, e.g, 38 months, 50 months, 52 months.

In one embodiment, the TNFα inhibitor, e.g, antibody, or an antigen-binding portion thereof, may also be administered to a subject for the treatment of bone loss for a period defined in years, e.g., 1 year, 2 years, 3 years, 4 years, 5 years, 6 years, 7 years, 8 years, etc. Ranges of values between any of the above recited values are also intended to be included in the scope of the invention, e.g, 1.5 years, 2.2 years, 3.5 years.

The TNFα inhibitor used in the method of the invention may also be administered according to a dosing determination known in the art. For example, in one embodiment, the TNFα inhibitor is administered to the subject for the treatment of bone loss according to a weight based dosing scheme, i.e., mg/kg whereby the amount of TNFα inhibitor is determined by the weight of the subject. Alternatively, the TNFα inhibitor may be administered according to a fixed dose or total body dose, whereby a constant fixed amount of TNFα inhibitor is delivered with each administration. In one embodiment, a human TNFα antibody, or antigen-binding portion thereof, is administered to the subject at a fixed dose ranging from 10-100 mg. In one embodiment, a human TNFα antibody, or antigen-binding portion thereof, is administered to the subject in a fixed dose of 40 mg, 50 mg, 60 mg, 70 mg, 80 mg, 90 mg, 100 mg, etc. Ranges of values between any of the above recited values are also intended to be included in the scope of the invention, e.g., 85 mg, 95 mg, as are ranges based on the aforementioned doses, e.g., 20-80 mg.

In one embodiment, administration of the TNFα inhibitor is parenteral (e.g., intravenous, subcutaneous, intraperitoneal, intramuscular). In one embodiment, the TNFα inhibitor is administered by intravenous infusion or injection. In another embodiment, the TNFα inhibitor is administered by intramuscular injection, or by subcutaneous injection (e.g., a biweekly, subcutaneous injection). In one embodiment, a human TNFα antibody, or antigen-binding portion thereof, is administered to the subject according to pulmonary techniques.

Dosage regimens described herein may be adjusted to provide the optimum desired response, e.g., treatment of bone loss, in consideration of the teachings herein. It is to be noted that dosage values may vary with the type and severity of bone loss. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention.

In one embodiment, the methods of the invention comprise selecting a subject having or at risk of having bone loss (or a disorder associated with bone loss). In another embodiment, the method of the invention comprises administering a TNFα inhibitor to a subject who was previously selected as having or was selected as at risk of having bone loss (or a disorder associated with bone loss).

In one embodiment, predictors of hand bone loss are described in the examples described herein, and include age and/or CRP levels.

The methods and compositions of the invention may be used to treat bone loss associated with another disorder. In one embodiment, a TNFα inhibitor is used to reduce bone loss, e.g., hand bone loss, in a subject having a disorder associated with bone loss. It is also within the scope of the invention that the methods described herein may be used to prevent bone loss in a subject having or at risk of having a disorder associated with bone loss. Additional details regarding disorders associated with bone loss are described below.

### Rheumatoid Arthritis

Tumor necrosis factor (TNF) is a pivotal cytokine in the pathogenesis of rheumatoid arthritis (RA). TNFα has been implicated in activating tissue inflammation and causing joint destruction in rheumatoid arthritis (see e.g., Moeller, A., et al. (1990) Cytokine 2:162-169; U.S. Pat. No. 5,231,024 to Moeller et al.; European Patent Publication No. 260 610 BI by Moeller, A.; Tracey and Cerami, supra; Arend, W. P. and Dayer, J-M. (1995) Arth. Rheum. 38:151-160; Fava, R. A., et al. (1993) Clin. Exp. Immunol. 94:261-266). In addition to joint destruction, subjects with RA have local and generalized bone loss.

In recent years biologic response modifiers that inhibit TNF activity have become established therapies for RA. Adalimumab, etanercept, and infliximab have demonstrated marked improvements in both disease control and delay and prevention of radiographic damage among RA patients, (Breedveld et al, Arthritis Rheum 2006; 54:26-37; Genovese et al J Rheumatol 2005; 32:1232-42; Keystone et al, Arthritis Rheum 2004; 50:1400-11; Navarro-Sarabia et al, Cochrane Database Syst Rev 2005 Jul. 20; (3):CD005113; Smolen et al, Arthritis Rheum 2006; 54:702-10; St. Clair et al Arthritis Rheum 2004; 50:3432-43; van der Heijde et al, Arthritis Rheum 2006; 54:1063-74).

With respect to the effects of anti-TNF therapy on hand bone loss in subjects having rheumatoid arthritis (RA), only a few studies have been conducted. One open-cohort study explored hand BMD change among RA patients receiving infliximab. In this cohort, a key finding was that, even if bone loss in hip and spine was arrested, hand bone loss was not halted. [9] Thus, although treatment with the TNFα inhibitor infliximab arrested generalized bone loss in subjects with RA, infliximab failed to arrest bone loss in the hands. The present invention provides the surprising discovery that TNFα inhibitors, such as human TNFα antibodies, may be used to treat bone loss, including hand bone loss, in subjects having RA.

It is also within the scope of the invention that TNFα inhibitors may be used to treat bone loss in subjects at risk of having bone loss, including, for example, subjects diagnosed with RA. In one embodiment, a subject at risk of developing bone loss is a subject having early RA, or diagnosed with RA for less than 3 years.

In one embodiment, treatment of bone loss is achieved by administering a human TNFα antibody, or an antigen-binding portion thereof, to a subject having rheumatoid arthritis, wherein the human TNFα antibody, or an antigen-binding portion thereof, is administered on a biweekly dosing regimen. In one embodiment, the human TNFα antibody, or an antigen-binding portion thereof, is administered in a dose of about 10-100 mg, including, but not limited to a dose of about 40 mg, 50 mg, 60 mg, 70 mg, 80 mg, 90 mg, or 100 mg. In one embodiment, the human TNFα antibody, or an antigen-binding portion thereof, is adalimumab or golimumab.

Surprisingly, as shown herein, it has been discovered that a human TNFα antibody, or an antigen-binding portion, reduces bone loss, e.g., cortical bone loss, e.g., cortical bone loss of the hand, in subjects with rheumatoid arthritis (RA) independently of its affect on clinically assessed disease activity. As such, benefits of TNFα inhibitor therapy may be derived in subjects having RA who do not show clinical improvements, as bone loss may be treated independently of clinical parameters.

### Osteoporosis

The methods of the invention may be used to treat bone loss, e.g., hand bone loss, in a subject having or at risk of having osteoporosis. Osteoporosis is a disease characterized by low bone mass and structural deterioration of bone tissue. Osteoporosis can lead to bone fragility and an increased susceptibility to fractures, especially of the hip, spine and wrist, although any bone can be affected. Examples of osteoporosis include, but are not limited to, idiopathic osteoporosis, secondary osteoporosis, and transient osteoporosis of the hip.

Osteopenia is a condition where bone mineral density is lower than normal, and can also be treated according to the methods of the invention. Osteopenia is often considered a precursor to osteoporosis. As such, TNFα inhibitors may be used to reduce or prevent bone loss, including hand bone loss, in patients having osteopenia.

Osteoporosis and osteopenia can result not only from aging and reproductive status, but can also be secondary to numerous diseases and disorders, as well as due to prolonged use of numerous medications, e.g., anticonvulsants (e.g., for epilepsy), corticosteroids (e.g., for rheumatoid arthritis and asthma), and/or immunosuppressive agents (e.g., for cancer). For example, glucocorticoid-induced osteoporosis is a form of osteoporosis that is caused by taking glucocorticoid medications such as prednisone (Deltasone, Orasone, etc.), prednisolone (Prelone), dexamethasone (Decadron, Hexadrol), and cortisone (Cortone Acetate). These medications are frequently used to help control many rheumatic diseases, including rheumatoid arthritis, systemic lupus erythematosus, inflammatory bowel disease, and polymyalgia rheumatica. Other diseases in which osteoporosis may be secondary include, but are not limited to, juvenile rheumatoid arthritis, diabetes, osteogenesis imperfecta, hyperthyroidism, hyperparathyroidism, Cushing's syndrome, malabsorption syndromes, anorexia nervosa and/or kidney disease. In addition, numerous behaviors have been associated with osteoporosis, such as, prolonged inactivity or immobility, inadequate nutrition (especially calcium, vitamin D), excessive exercise leading to amenorrhea (absence of periods), smoking, and/or alcohol abuse.

Notably, patients with rheumatologic disorders like rheumatoid arthritis, ankylosing spondylitis, systemic lupus erythematosus, and polyarticular juvenile idiopathic arthritis are at increased risk of osteoporosis, either as part of their disease or because of other risk factors (notably corticosteroid therapy). As such, in one embodiment, methods of the invention may be used to treat osteoporosis in a patient having rheumatoid arthritis.

### Osteoarthritis

The methods of the invention may be used to treat bone loss, e.g., hand bone loss, in a subject having or at risk of having osteoarthritis. Tumor necrosis factor has been implicated in the pathophysiology of osteoarthritis, (Venn et al. (1993) Arthritis Rheum. 36:819; Westacott et al. (1994) J Rheumatol. 21:1710). Osteoarthritis (OA) is also referred to as hypertrophic osteoarthritis, osteoarthrosis, and degenerative joint disease. OA is a chronic degenerative disease of skeletal joints, which affects specific joints, commonly knees, hips, hand joints and spine, in adults of all ages. OA is characterized by a number of the following manifestations including degeneration and thinning of the articular cartilage with associated development of “ulcers” or craters, osteophyte formation, hypertrophy of bone at the margins, and changes in the snyovial membrane and enlargement of affected joints. Furthermore, osteoarthritis is accompanied by pain and stiffness, particularly after prolonged activity. The antibody, or antigen-binding fragment thereof, of the invention can be used to treat osteoarthritis. Characteristic radiographic features of osteoarthritis include joint space narrowing, subchondral sclerosis, osteophytosis, subchondral cyst formation, loose osseous body (or “joint mouse”).

Medications used to treat osteoarthritis include a variety of nonsteroidal, anti-inflammatory drugs (NSAIDs). In addition, COX 2 inhibitors, including Celebrex, Vioxx, and Bextra, and Etoricoxib, are also used to treat OA. Steroids, which may be injected directly into the joint, may also be used to reduce inflammation and pain. In one embodiment of the invention, TNFα antibodies of the invention are administered in combination with an NSAID, a COX2 inhibitor, and/or steroids.

Other Disorders Associated with Bone Loss

In another embodiment, bone loss may be treated in a subject having or at risk of having a disorder associated with bone loss, i.e., a disorder in which there is progressive loss of bone density and thinning of bone tissue. Such conditions include, but are not limited to, erosive arthritis, bone malignancies, osteomalacia, skeletal changes of hyperparathyroidism, chronic renal failure (renal osteodystrophy), osteitis deformans (Paget's disease of bone), and osteolytic metastases. In one embodiment, the methods of the invention are used to treat a subject having a TNFα-related disorder (see, for example, disorders described in US20040126372 and U.S. Pat. No. 6,090,382, the contents of each of which are expressly incorporated herein by reference).

In one embodiment, the subject who is administered a TNFα inhibitor for the treatment of bone loss, may be selected for having and/or being at risk of having bone loss. For example, a subject who is postmenopausal may be at risk of developing bone loss. In another example, a subject diagnosed with osteoarthritis may have bone loss, including hand bone loss, and, therefore, may benefit from the methods of the invention. Thus, in one embodiment, the invention includes identifying subjects who may benefit from the methods of the invention, i.e., treatment of bone loss, e.g., hand bone loss, and subsequently administering a TNFα inhibitor to the subject for treatment. In one embodiment, the invention also provides a method for treating hand bone loss in a subject, comprising selecting a subject who has hand bond loss or is at risk of having hand bone loss and administering a TNFα inhibitor to the subject, such that hand bone loss is treated. Alternatively, the method of the invention may be performed on a subject who was previously selected as having or at risk of having bone loss, including hand bone loss.

### III. TNF Inhibitors

A TNFα inhibitor used in the methods and compositions of the invention includes any agent which interferes with TNFα activity. In a preferred embodiment, the TNFα inhibitor can neutralize TNFα activity, particularly detrimental TNFα activity.

In one embodiment, the TNFα inhibitor used in the invention is an TNFα antibody (also referred to herein as a TNFα antibody), or an antigen-binding fragment thereof, including chimeric, humanized, and human antibodies. Examples of TNFα antibodies which may be used in the invention include, but not limited to, infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502 and U.S. Pat. No. 7,250,165, incorporated by reference herein), and adalimumab (HUMIRA® Abbott Laboratories, a human anti-TNF mAb, described in U.S. Pat. No. 6,090,382 as D2E7). Additional TNF antibodies (and sequences thereof) which may be used in the invention are described in U.S. Pat. Nos. 6,593,458; 6,498,237; 6,451,983; 7,250,165; and 6,448,380, each of which is expressly incorporated by reference herein.

Other examples of TNFα inhibitors which may be used in the methods and compositions of the invention include etanercept (Enbrel, described in WO 91/03553 and WO 09/406,476), soluble TNF receptor Type I, a pegylated soluble TNF receptor Type I (PEGs TNF-R1), p55TNFR1gG (Lenercept), and recombinant TNF binding protein (r-TBP-I) (Serono).

In one embodiment, the term “TNFα inhibitor” excludes infliximab. In one embodiment, the term “TNFα inhibitor” excludes adalimumab. In another embodiment, the term “TNFα inhibitor” excludes adalimumab and infliximab.

In one embodiment, the term “TNFα inhibitor” excludes etanercept, and, optionally, adalimumab, infliximab, and adalimumab and infliximab.

In one embodiment, the term “TNFα antibody” excludes infliximab. In one embodiment, the term “TNFβ antibody” excludes adalimumab. In another embodiment, the term “TNFα antibody” excludes adalimumab and infliximab.

In one embodiment, the invention features uses and composition for treating or determining the efficacy of a TNFα inhibitor for the treatment of bone loss, wherein the TNFα antibody is an isolated human antibody, or antigen-binding portion thereof, that binds to human TNFα with high affinity and a low off rate, and also has a high neutralizing capacity. Preferably, the human antibodies used in the invention are recombinant, neutralizing human anti-hTNFα antibodies. The most preferred recombinant, neutralizing antibody of the invention is referred to herein as D2E7, also referred to as HUMIRA® or adalimumab (the amino acid sequence of the D2E7 VL region is shown in SEQ ID NO: 1; the amino acid sequence of the D2E7 VH region is shown in SEQ ID NO: 2). The properties of D2E7 (adalimumab/HUMIRA®) have been described in Salfeld et al., U.S. Pat. Nos. 6,090,382, 6,258,562, and 6,509,015, which are each incorporated by reference herein. The methods of the invention may also be performed using chimeric and humanized murine anti-hTNFα antibodies which have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342).

In one embodiment, the method of the invention includes determining the efficacy of D2E7 antibodies and antibody portions, D2E7-related antibodies and antibody portions, or other human antibodies and antibody portions with equivalent properties to D2E7, such as high affinity binding to hTNFα with low dissociation kinetics and high neutralizing capacity, for the treatment of bone loss. In one embodiment, the invention provides treatment with an isolated human antibody, or an antigen-binding portion thereof, that dissociates from human TNFα with a Kd of 1×10-8 M or less and a Koff rate constant of 1×10-3 s−1 or less, both determined by surface plasmon resonance, and neutralizes human TNFα cytotoxicity in a standard in vitro L929 assay with an IC50 of 1×10-7 M or less. More preferably, the isolated human antibody, or antigen-binding portion thereof, dissociates from human TNFα with a Koff of 5×10-4 s⁻¹ or less, or even more preferably, with a Koff of 1×10-4 s⁻¹ or less. More preferably, the isolated human antibody, or antigen-binding portion thereof, neutralizes human TNFα cytotoxicity in a standard in vitro L929 assay with an IC50 of 1×10-8 M or less, even more preferably with an IC50 of 1×10-9 M or less and still more preferably with an IC50 of 1×10-10 M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating bone loss by administering human antibodies that have slow dissociation kinetics for association with hTNFα and that have light and heavy chain CDR3 domains that structurally are identical to or related to those of D2E7. Position 9 of the D2E7 VL CDR3 can be occupied by Ala or Thr without substantially affecting the Koff. Accordingly, a consensus motif for the D2E7 VL CDR3 comprises the amino acid sequence: Q-R—Y—N—R-A-P—Y-(T/A) (SEQ ID NO: 3). Additionally, position 12 of the D2E7 VH CDR3 can be occupied by Tyr or Asn, without substantially affecting the Koff. Accordingly, a consensus motif for the D2E7 VH CDR3 comprises the amino acid sequence: V—S—Y-L-S-T-A-S—S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the Koff. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 appear to be critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382).

Accordingly, in another embodiment, the antibody or antigen-binding portion thereof preferably contains the following characteristics:
a) dissociates from human TNFα with a Koff rate constant of 1×10-3 s-1 or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a Koff of 5×10-4 s-1 or less. Even more preferably, the antibody, or antigen-binding portion thereof, dissociates from human TNFα with a Koff of 1×10-4 s⁻¹ or less.

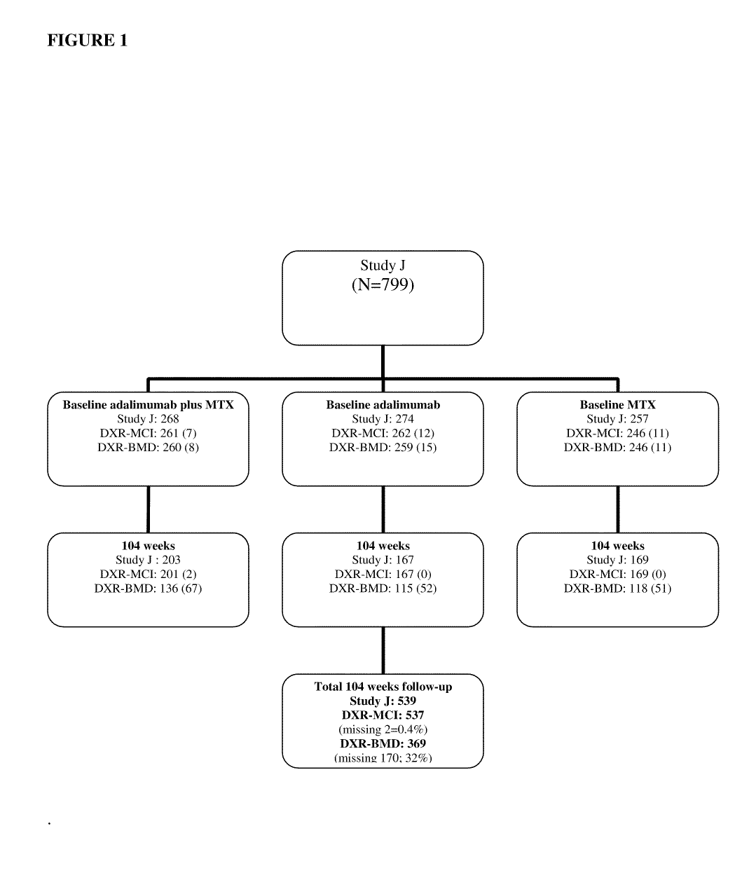

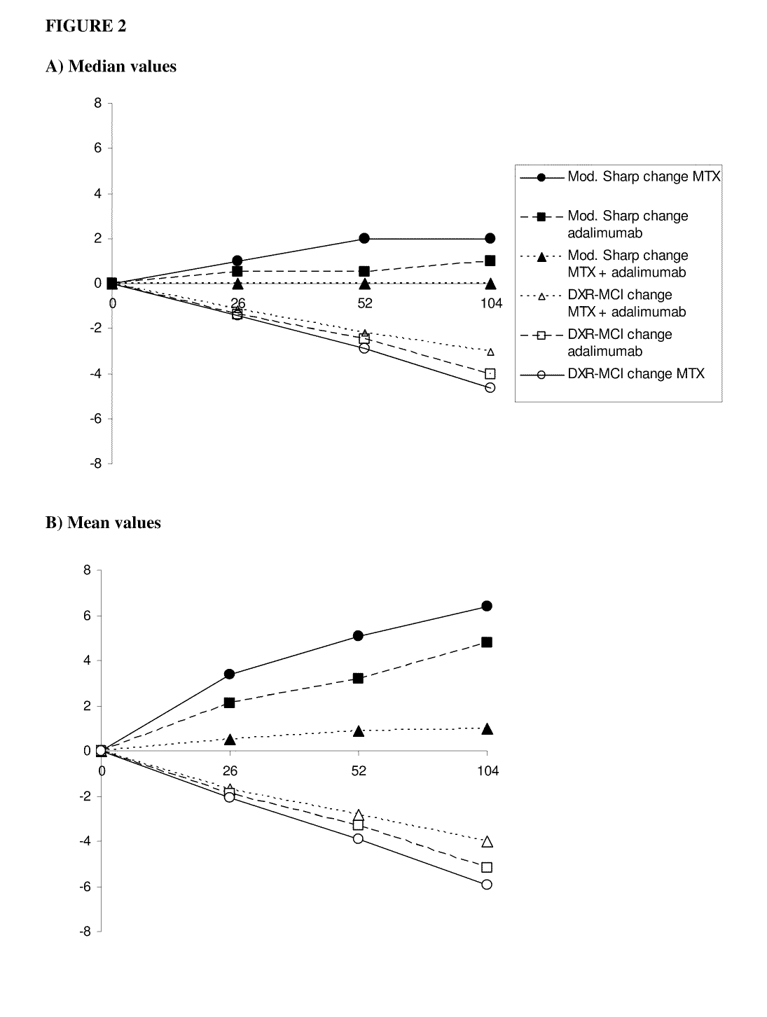

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. Preferably, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). Even more preferably, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the VκI human germline family, more preferably from the A20 human germline Vk gene and most preferably from the D2E7 VL framework sequences shown in FIGS. 1A and 1B of U.S. Pat. No. 6,090,382. The framework regions for VH preferably are from the VH3 human germline family, more preferably from the DP-31 human germline VH gene and most preferably from the D2E7 VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portions thereof, containing D2E7-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

The TNFα antibody used in the methods and compositions of the invention may be modified for improved treatment of bone loss. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivative other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to educe or prevent bone loss by administration of the TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express adalimumab (D2E7) or an adalimumab (D2E7)-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline VH Sequences Reveals about Fifty Groups of VH Segments with Different Hypervariable Loops” J. Mol. Biol. 227:776-798; and Cox, J. P. L. et al. (1994) “A Directory of Human Germ-line V78 Segments Reveals a Strong Bias in their Usage” Eur. J. Immunol. 24:827-836; the contents of each of which are expressly incorporated herein by reference). To obtain a DNA fragment encoding the heavy chain variable region of D2E7, or a D2E7-related antibody, a member of the VH3 family of human germline VH genes is amplified by standard PCR. Most preferably, the DP-31 VH germline sequence is amplified. To obtain a DNA fragment encoding the light chain variable region of D2E7, or a D2E7-related antibody, a member of the VκI family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the D2E7 or D2E7-related amino acid sequences disclosed herein. The amino acid sequences encoded by the germline VH and VL DNA sequences are first compared to the D2E7 or D2E7-related VH and VL amino acid sequences to identify amino acid residues in the D2E7 or D2E7-related sequence that differ from germline. Then, the appropriate nucleotides of the germline DNA sequences are mutated such that the mutated germline sequence encodes the D2E7 or D2E7-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding D2E7 or D2E7-related VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly-4-Ser)3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the D2E7 or D2E7-related light or heavy chain sequences, the expression vector may already carry antibody constant region sequences. For example, one approach to converting the D2E7 or D2E7-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr—CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNFα antibody adalimumab (D2E7). The nucleotide sequence encoding the D2E7 light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding D2E7-related antibodies, or portions thereof (e.g., a CDR domain, such as a CDR3 domain), can be derived from the nucleotide sequences encoding the D2E7 LCVR and HCVR using the genetic code and standard molecular biology techniques.

Recombinant human antibodies of the invention in addition to D2E7 or an antigen binding portion thereof, or D2E7-related antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating and screening antibody display libraries can be found in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-65; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982.

In a preferred embodiment, to isolate human antibodies with high affinity and a low off rate constant for hTNFα, a murine anti-hTNFα antibody having high affinity and a low off rate constant for hTNFα (e.g., MAK 195, the hybridoma for which has deposit number ECACC 87 050801) is first used to select human heavy and light chain sequences having similar binding activity toward hTNFα, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J. 12:725-734. The scFv antibody libraries preferably are screened using recombinant human TNFα as the antigen.

Once initial human VL and VH segments are selected, “mix and match” experiments, in which different pairs of the initially selected VL and VH segments are screened for hTNFα binding, are performed to select preferred VL/VH pair combinations. Additionally, to further improve the affinity and/or lower the off rate constant for hTNFα binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to hTNFα and sequences that exhibit high affinity and a low off rate for hTNFα binding can be selected.

Following screening and isolation of an anti-hTNFα antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in above.

Methods of isolating human neutralizing antibodies with high affinity and a low off rate constant for hTNFα are described in U.S. Pat. Nos. 6,090,382, 6,258,562, and 6,509,015, each of which is incorporated by reference herein.

Antibodies, antibody-portions, and other TNFα inhibitors for use in the methods of the invention, can be incorporated into pharmaceutical compositions suitable for administration to a subject. Typically, the pharmaceutical composition comprises an antibody, antibody portion, or other TNFα inhibitor, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion, or other TNFα inhibitor.

The compositions for use in the methods and compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies or other TNFα inhibitors. The preferred mode of administration is parenteral (e.g., intravenous, subcutaneous, intraperitoneal, intramuscular). In a preferred embodiment, the antibody or other TNFα inhibitor is administered by intravenous infusion or injection. In another preferred embodiment, the antibody or other TNFα inhibitor is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the invention includes pharmaceutical compositions comprising an effective TNFα inhibitor and a pharmaceutically acceptable carrier, wherein the effective TNFα inhibitor may be used to treat rheumatoid arthritis.

In one embodiment, the antibody or antibody portion for use in the methods of the invention is incorporated into a pharmaceutical formulation as described in PCT/IB03/04502 and U.S. Appln. No. 20040033228, incorporated by reference herein. This formulation includes a concentration 50 mg/ml of the antibody D2E7 (adalimumab), wherein one pre-filled syringe contains 40 mg of antibody for subcutaneous injection.

The antibodies, antibody-portions, and other TNFα inhibitors of the present invention can be administered by a variety of methods known in the art, although for many therapeutic applications, the preferred route/mode of administration is parenteral, e.g., subcutaneous injection. In another embodiment, administration is via intravenous injection or infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, Robinson, ed., Dekker, Inc., New York, 1978.

In one embodiment, the TNFα antibodies and inhibitors used in the invention are delivered to a subject subcutaneously. In one embodiment, the subject administers the TNFα inhibitor, including, but not limited to, TNFα antibody, or antigen-binding portion thereof, to himself/herself.

TNFα antibodies and inhibitors used in the invention may also be administered in the form of protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the TNFα antibodies of the invention are delivered via a protein delivery system, wherein one or more of a protein crystal formulation or composition, is administered to a subject with a TNFα-related disorder. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. Appln. No. 20040033228, incorporated by reference herein, are used to treat rheumatoid arthritis using the treatment methods of the invention.

In certain embodiments, an antibody, antibody portion, or other TNFα inhibitor of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion for use in the methods of the invention is coformulated with and/or coadministered with one or more additional therapeutic agents, including a rheumatoid arthritis inhibitor or antagonist. For example, an anti-hTNFα antibody or antibody portion of the invention may be coformulated and/or coadministered with one or more additional antibodies that bind other targets associated with TNFα related disorders (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Additional description regarding methods and uses of the invention comprising administration of a TNFα inhibitor are also described in Part II of this specification.

The invention also pertains to packaged pharmaceutical compositions or kits for administering the anti-TNF antibodies of the invention for the treatment of rheumatoid arthritis. In one embodiment of the invention, the kit comprises a TNFα inhibitor, such as an antibody and instructions for administration of the TNFα inhibitor for treating bone loss. The instructions may describe how, e.g., subcutaneously, and when, e.g., at week 0, week 2, week 4, etc., the different doses of TNFα inhibitor shall be administered to a subject for treatment.

Another aspect of the invention pertains to kits containing a pharmaceutical composition comprising a TNFα inhibitor, such as an antibody, and a pharmaceutically acceptable carrier and one or more pharmaceutical compositions each comprising an additional therapeutic agent useful for treating bone loss, and a pharmaceutically acceptable carrier. Alternatively, the kit comprises a single pharmaceutical composition comprising an anti-TNFα antibody, one or more drugs useful for treating bone loss, and a pharmaceutically acceptable carrier. The instructions may describe how, e.g., subcutaneously, and when, e.g., at week 0, week 2, week 4, etc., the different doses of TNFα inhibitor and/or the additional therapeutic agent shall be administered to a subject for treatment.

The kit may contain instructions for dosing of the pharmaceutical compositions for the treatment of bone loss. Additional description regarding articles of manufacture of the invention are described in subsection II.

The package or kit alternatively can contain the TNFα inhibitor and it can be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### IV. Articles of Manufacture

The invention also provides a packaged pharmaceutical composition wherein the TNFα inhibitor, e.g., TNFα antibody, is packaged within a kit or an article of manufacture. The kit or article of manufacture of the invention contains materials useful for the treatment, including induction and/or remission, prevention and/or diagnosis of bone loss. The kit or article of manufacture comprises a container and a label or package insert or printed material on or associated with the container which provides information regarding use of the TNFα inhibitor, e.g., a TNFα antibody, for the treatment of bone loss.

A kit or an article of manufacture refers to a packaged product comprising components with which to administer a TNFα inhibitor for treatment of bone loss. The kit preferably comprises a box or container that holds the components of the kit. The box or container is affixed with a label or a Food and Drug Administration approved label, including a protocol for administering the TNFα inhibitor. The box or container holds components of the invention which are preferably contained within plastic, polyethylene, polypropylene, ethylene, or propylene vessels. The vessels can be capped-tubes or bottles. The kit can also include instructions for administering the TNFα antibody of the invention. In one embodiment the kit of the invention includes the formulation comprising the human antibody adalimumab (or D2E7), as described in PCT/IB03/04502 and U.S. application Ser. No. 10/222,140, incorporated by reference herein.

The term “package insert” is used to refer to instructions customarily included in commercial packages of therapeutic products, that contain information about the indications, usage, dosage, administration, contraindications and/or warnings concerning the use of such therapeutic products.

In one embodiment, the article of manufacture of the invention comprises (a) a first container with a composition contained therein, wherein the composition comprises a TNFα antibody; and (b) a package insert indicating that the TNFα antibody may be used for treating bone loss.

Suitable containers for the TNFα inhibitor, e.g., a TNFα antibody, include, for example, bottles, vials, syringes, pens, etc. The containers may be formed from a variety of materials such as glass or plastic. The container holds a composition which is by itself or when combined with another composition effective for treating, preventing and/or diagnosing the condition and may have a sterile access port.

In one embodiment, the article of manufacture comprises a TNFα inhibitor, e.g., a TNFα antibody, and a label which indicates to a subject who will be administering the TNFα inhibitor about using the TNFα inhibitor for the treatment of bone loss. The label may be anywhere within or on the article of manufacture. In one embodiment, the article of manufacture comprises a container, such as a box, which comprises the TNFα inhibitor and a package insert or label providing information pertaining to use of the TNFα inhibitor for the treatment of bone loss. In another embodiment, the information is printed on a label which is on the outside of the article of manufacture, in a position which is visible to prospective purchasers.

In one embodiment, the package insert of the invention informs a reader, including a subject, e.g., a purchaser, who will be administering the TNFα inhibitor for treatment, that the TNFα inhibitor, e.g., a TNFα antibody such as adalimumab, is an indicated treatment of bone loss.

The package insert of the invention may also provide information to subjects who will be receiving adalimumab regarding combination uses for both safety and efficacy purposes. The package insert of the invention may contain warnings and precautions regarding the use of the TNFα inhibitor, e.g., a TNFα antibody such as adalimumab. In one embodiment, the invention provides an article of manufacture comprising a packaging material; a TNFα antibody, or antigen-binding portion thereof; and a label or package insert contained within the packaging material indicating that in studies of the TNFα antibody, or antigen-binding portion thereof, certain adverse events were observed, including any of those described in the Examples.

The label of the invention may contain information regarding the use of the TNFα inhibitor, e.g., a TNFα antibody such as adalimumab, in clinical studies for bone loss. In one embodiment, the label of the invention describes the studies described herein as the Examples, either as a whole or in portion.

In one embodiment of the invention, the kit comprises a TNFα inhibitor, such as an antibody, a second pharmaceutical composition comprising an additional therapeutic agent, and instructions for administration of both agents for the treatment of bone loss. The instructions may describe how, e.g., subcutaneously, and when, e.g., at week 0, week 2, and biweekly thereafter, doses of TNFα antibody and/or the additional therapeutic agent shall be administered to a subject for treatment.

Another aspect of the invention pertains to kits containing a pharmaceutical composition comprising an anti-TNFα antibody and a pharmaceutically acceptable carrier and one or more additional pharmaceutical compositions each comprising a drug useful for treating a TNFα related disorder and a pharmaceutically acceptable carrier. Alternatively, the kit comprises a single pharmaceutical composition comprising an anti-TNFα antibody, one or more drugs useful for treating a TNFα related disorder and a pharmaceutically acceptable carrier. The kits further contain instructions for dosing of the pharmaceutical compositions for the treatment of a TNFα related disorder.

The package or kit alternatively may contain the TNFα inhibitor and it may be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### V. Additional Therapeutic Agents

Methods, uses, and compositions of the invention also include combinations of TNFα inhibitors, including antibodies, and other therapeutic agents for the treatment of bone loss, including, but not limited to, hand bone loss. It should be understood that the antibodies of the invention or antigen binding portion thereof can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent that imparts a beneficial attribute to the therapeutic composition e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

In one embodiment, a TNFα inhibitor is administered in combination with an antiresorptive agent, including, but not limited to, alendronate, alendronate plus vitamin D3, ibandronate, risedronate, risedronate with calcium, zoledronic acid, calcitonin, estrogen, and, raloxifene. In yet another embodiment, the TNFα inhibitor is administered in combination with a bone forming agent, such as a parathyroid hormone, e.g., teriparatide.

TNFα inhibitors described herein may be used in combination with additional therapeutic agents such as a Disease Modifying Anti-Rheumatic Drug (DMARD) or a Nonsteroidal Antiinflammatory Drug (NSAID) or a steroid or any combination thereof. Preferred examples of a DMARD are hydroxychloroquine, leflunomide, methotrexate, parenteral gold, oral gold and sulfasalazine. Preferred examples of non-steroidal anti-inflammatory drug(s) also referred to as NSAIDS include drugs like ibuprofen. Other preferred combinations are corticosteroids including prednisolone; the well known side effects of steroid use can be reduced or even eliminated by tapering the steroid dose required when treating patients in combination with the anti-TNFα antibodies of this invention. Non-limiting examples of therapeutic agents for rheumatoid arthritis with which an antibody, or antibody portion, of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, IL-23, interferons, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNFα inhibitors such as soluble p55 or p75 TNF receptors, derivatives, thereof, (p75TNFR1gG (Enbrel™) or p55TNFR1gG (Lenercept), chimeric, humanized or human TNF antibodies, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (Humira® Abbott Laboratories, a human anti-TNF mAb, described in U.S. Pat. No. 6,090,382 as D2E7). Additional TNF antibodies which can be used in the invention are described in U.S. Pat. Nos. 6,593,458; 6,498,237; 6,451,983; and 6,448,380, each of which is incorporated by reference herein. Other combinations including TNFα converting enzyme (TACE) inhibitors; IL-1 inhibitors (Interleukin-1-converting enzyme inhibitors, IL-1RA etc.) may be effective for the same reason. Other combinations include the IL-6 antibody tocilizumab (Actemra). Other preferred combinations include Interleukin 11. Yet another preferred combination are other key players of the autoimmune response which may act parallel to, dependent on or in concert with TNFα function; especially preferred are IL-18 antagonists including IL-18 antibodies or soluble IL-18 receptors, or IL-18 binding proteins. It has been shown that TNFα and IL-18 have overlapping but distinct functions and a combination of antagonists to both may be most effective. Yet another preferred combination are non-depleting anti-CD4 inhibitors. Yet other preferred combinations include antagonists of the co-stimulatory pathway CD80 (B7.1) or CD86 (B7.2) including antibodies, soluble receptors or antagonistic ligands.

In one embodiment, the methods and compositions of the invention provide a combination use of a TNFα antibody, e.g., adalimumab, and a DMARD, e.g., methotrexate.

The TNFα inhibitors used in the methods and compositions of the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNFα or IL-1 (e.g. IRAK, NIK, IKK, p38 or MAP kinase inhibitors), IL-1β converting enzyme inhibitors, TNFα converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFR1gG (Enbrel and p55TNFR1gG (Lenercept)), sIL-1RI, sIL-1RI, sIL-6R), antiinflammatory cytokines (e.g. IL-4, IL-10, IL-11, IL-13 and TGFβ), tocilizumab (Actemra), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, and Mesopram. Preferred combinations include methotrexate or leflunomide and in moderate or severe rheumatoid arthritis cases, cyclosporine.

Nonlimiting additional agents may also be used in combination with a TNFα inhibitor to treat a disorder associated with detrimental TNFα activity and bone loss. For example, included within the scope of the invention is the combination use of a TNFα antibody, or antigen-binding portion thereof, and an agent for treating rheumatoid arthritis, including, but not limited to, the following: non-steroidal anti-inflammatory drug(s) (NSAIDs); cytokine suppressive anti-inflammatory drug(s) (CSAIDs); CDP-571/BAY-10-3356 (humanized anti-TNFα antibody; Celltech/Bayer); cA2/infliximab (chimeric anti-TNFα antibody; Centocor); 75 kdTNFR-IgG/etanercept (75 kD TNF receptor-IgG fusion protein; Immunex; see e.g., Arthritis & Rheumatism (1994) Vol. 37, S295; J. Invest. Med. (1996) Vol. 44, 235A); 55 kdTNF-IgG (55 kD TNF receptor-IgG fusion protein; Hoffmann-LaRoche); IDEC-CE9.1/SB 210396 (non-depleting primatized anti-CD4 antibody; IDEC/SmithKiine; see e.g., Arthritis & Rheumatism (1995) Vol. 38, S185); DAB 486-IL-2 and/or DAB 389-IL-2 (IL-2 fusion proteins; Seragen; see e.g., Arthritis & Rheumatism (1993) Vol. 36, 1223); Anti-Tac (humanized anti-IL-2Rα; Protein Design Labs/Roche); IL-4 (anti-inflammatory cytokine; DNAX/Schering); IL-10 (SCH 52000; recombinant IL-10, anti-inflammatory cytokine; DNAX/Schering); IL-4; IL-10 and/or IL-4 agonists (e.g., agonist antibodies); IL-IRA (IL-1 receptor antagonist; Synergen/Amgen); anakinra (Kineret®/Amgen); TNF-bp/s-TNF (soluble TNF binding protein; see e.g., Arthritis & Rheumatism (1996) Vol. 39, No. 9 (supplement), S284; Amer. J. Physiol.—Heart and Circulatory Physiology (1995) Vol. 268, pp. 37-42); R973401 (phosphodiesterase Type IV inhibitor; see e.g., Arthritis & Rheumatism (1996) Vol. 39, No. 9 (supplement), S282); MK-966 (COX-2 Inhibitor; see e.g., Arthritis & Rheumatism (1996) Vol. 39, No. 9 (supplement), S81); Iloprost (see e.g., Arthritis & Rheumatism (1996) Vol. 39, No. 9 (supplement), S82); methotrexate; thalidomide (see e.g., Arthritis & Rheumatism (1996) Vol. 39, No. 9 (supplement), S282) and thalidomide-related drugs (e.g., Celgen); leflunomide (anti-inflammatory and cytokine inhibitor; see e.g., Arthritis & Rheumatism (1996) Vol. 39, No. 9 (supplement), S131; Inflammation Research (1996) Vol. 45, pp. 103-107); tranexamic acid (inhibitor of plasminogen activation; see e.g., Arthritis & Rheumatism (1996) Vol. 39, No. 9 (supplement), S284); T-614 (cytokine inhibitor; see e.g., Arthritis & Rheumatism (1996) Vol. 39 No. 9 (supplement), S282); prostaglandin E1 (see e.g., Arthritis & Rheumatism (1996) Vol. 39, No. 9 (supplement), S282); Tenidap (non-steroidal anti-inflammatory drug; see e.g., Arthritis & Rheumatism (1996) Vol. 39, No. 9 (supplement), S280); Naproxen (non-steroidal anti-inflammatory drug; see e.g., Neuro Report (1996) Vol. 7, pp. 1209-1213); Meloxicam (non-steroidal anti-inflammatory drug); Ibuprofen (non-steroidal anti-inflammatory drug); Piroxicam (non-steroidal anti-inflammatory drug); Diclofenac (non-steroidal anti-inflammatory drug); Indomethacin (non-steroidal anti-inflammatory drug); Sulfasalazine (see e.g., Arthritis & Rheumatism (1996) Vol. 39, No. 9 (supplement), S281); Azathioprine (see e.g., Arthritis & Rheumatism (1996) Vol. 39 No. 9 (supplement), S281); ICE inhibitor (inhibitor of the enzyme interleukin-1β converting enzyme); zap-70 and/or lck inhibitor (inhibitor of the tyrosine kinase zap-70 or lck); VEGF inhibitor and/or VEGF-R inhibitor (inhibitors of vascular endothelial cell growth factor or vascular endothelial cell growth factor receptor; inhibitors of angiogenesis); corticosteroid anti-inflammatory drugs (e.g., SB203580); TNF-convertase inhibitors; anti-IL-12 antibodies; anti-IL-18 antibodies; interleukin-11 (see e.g., Arthritis & Rheumatism (1996) Vol. 39, No. 9 (supplement), S296); interleukin-13 (see e.g., Arthritis & Rheumatism (1996) Vol. 39, No. 9 (supplement), S308); interleukin-17 inhibitors (see e.g., Arthritis & Rheumatism (1996) Vol. 39, No. 9 (supplement), S120); gold; penicillamine; chloroquine; chlorambucil; hydroxychloroquine; cyclosporine; cyclophosphamide; total lymphoid irradiation; anti-thymocyte globulin; anti-CD4 antibodies; CD5-toxins; orally-administered peptides and collagen; lobenzarit disodium; Cytokine Regulating Agents (CRAs) HP228 and HP466 (Houghten Pharmaceuticals, Inc.); ICAM-1 antisense phosphorothioate oligo-deoxynucleotides (ISIS 2302; Isis Pharmaceuticals, Inc.); soluble complement receptor 1 (TP10; T Cell Sciences, Inc.); prednisone; orgotein; glycosaminoglycan polysulphate; minocycline; anti-IL2R antibodies; marine and botanical lipids (fish and plant seed fatty acids; see e.g., DeLuca et al. (1995) Rheum. Dis. Clin. North Am. 21:759-777); auranofin; phenylbutazone; meclofenamic acid; flufenamic acid; intravenous immune globulin; zileuton; azaribine; mycophenolic acid (RS-61443); tacrolimus (FK-506); sirolimus (rapamycin); amiprilose (therafectin); cladribine (2-chlorodeoxyadenosine); methotrexate; antivirals; and immune modulating agents.

In one embodiment, a TNFα antibody, or antigen-binding portion thereof, is administered in combination with one of the following agents for the treatment of rheumatoid arthritis: small molecule inhibitor of KDR (ABT-123), small molecule inhibitor of Tie-2; methotrexate; prednisone; celecoxib; folic acid; hydroxychloroquine sulfate; rofecoxib; etanercept; infliximab; leflunomide; naproxen; valdecoxib; sulfasalazine; methylprednisolone; ibuprofen; meloxicam; methylprednisolone acetate; gold sodium thiomalate; aspirin; azathioprine; triamcinolone acetonide; propxyphene napsylate/apap; folate; nabumetone; diclofenac; piroxicam; etodolac; diclofenac sodium; oxaprozin; oxycodone hcl; hydrocodone bitartrate/apap; diclofenac sodium/misoprostol; fentanyl; anakinra, human recombinant; tramadol hcl; salsalate; sulindac; cyanocobalamin/fa/pyridoxine; acetaminophen; alendronate sodium; prednisolone; morphine sulfate; lidocaine hydrochloride; indomethacin; glucosamine sulfate/chondroitin; cyclosporine; amitriptyline hcl; sulfadiazine; oxycodone hcl/acetaminophen; olopatadine hcl; misoprostol; naproxen sodium; omeprazole; mycophenolate mofetil; cyclophosphamide; rituximab; IL-1 TRAP; MRA; CTLA4-IG; IL-18 BP; ABT-874; ABT-325 (anti-IL 18); anti-IL 15; BIRB-796; SCIO-469; VX-702; AMG-548; VX-740; Roflumilast; IC-485; CDC-801; and mesopram. In another embodiment, a TNF antibody, or antigen-binding portion thereof, is administered for the treatment of an TNF-related disorder in combination with one of the above mentioned agents for the treatment of rheumatoid arthritis.

The antibodies of the invention, or antigen binding portions thereof, may also be combined with agents, such as alemtuzumab, dronabinol, Unimed, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists, IL-4 agonists.

The present invention is further illustrated by the following example, which should not be construed as limiting in any way.

### Example

### Anti-TNFα Therapy Reduces Bone Loss

A. Adalimumab Therapy Reduces Hand Bone Loss in Patients with Early Rheumatoid Arthritis

### Overall Summary

One objective of this study (Study J) was to compare cortical hand bone loss in patients with early rheumatoid arthritis (RA) across the three treatment arms: adalimumab plus methotrexate (MTX); adalimumab monotherapy; and MTX monotherapy. A secondary aim was to search for predictors of hand bone loss.

Generally, the study included 768 patients with active RA for <3 years who were naïve to MTX. Clinical data collections and radiographic examinations of hands were performed at baseline, and after 26, 52 and 104 weeks of therapy. Hand bone loss was assessed by digital X-ray radiogrammetry (DXR), primarily as metacarpal cortical index (MCI), on the same radiographs used for joint damage assessments.

The results showed that the median percentage loss of DXR-MCI was significantly greater in the MTX group vs. the combination group at 52 weeks (−2.87 vs. −2.16, p=0.009) and 104 weeks (−4.62 vs. −3.03, p<0.001). Median loss in the MTX group was numerically greater than loss in the adalimumab group at 52 weeks (−2.87 vs. −2.45, p=0.19) and 104 weeks (−4.62 vs. −4.03, p=0.10). In addition, older age, elevated baseline CRP, and non-use of adalimumab were independent predictors of hand bone loss in a linear regression model.

In conclusion, Adalimumab protected against hand bone loss in early RA. The order of bone loss across the three treatment arms was similar to the order of radiographic progression. This analysis of data supports quantitative measures of hand bone for the detection of inflammatory bone damage in RA patients. It also demonstrates that hand bone loss and radiographic bone damage occur through similar pathogenetic mechanisms.

### Objective

The primary objective of this analysis was to compare cortical hand bone loss in the three arms of Study J: adalimumab plus methotrexate (MTX) vs. adalimumab monotherapy vs. MTX monotherapy, all for patients with early, aggressive RA. Secondarily, potential predictors of hand bone loss in the Study J RA patients were evaluated.

### Methods

### Study Sample and Design

The radiographic and clinical data from this 2-year, multi-centre, double-blind, randomised controlled study (Study J) was previously been described in detail (see Breedveld et al. (2006) Arthritis Rheum 54:26-37). The efficacy and safety of adalimumab plus MTX was compared with adalimumab monotherapy and with MTX monotherapy for 799 adult patients with early (<3 years), aggressive RA (mean erosion score of approximately 12 Sharp units, estimated annual TSS progression of approximately 27 Sharp units) who previously had not been treated with MTX, cyclophosphamide, cyclosporine, azathioprin or more than 2 other DMARDs (Breedveld et al. (2006), supra). The combination group received adalimumab 40 mg subcutaneously (sc) every other week plus weekly oral MTX (rapidly increased to 20 mg/week), and the monotherapy groups received either adalimumab 40 mg sc every other week plus placebo or weekly oral MTX plus placebo. Radiographs from hands and feet were scored according to a modified Sharp score (range 0-398) (Breedveld et al. (2006), supra).

The following study presents hand bone loss data for 26, 52, and 104 weeks of follow-up. To maintain the original study design of a blinded randomised controlled trial, the treatment code was kept secret for the investigator who analyzed the data, until all analyses had been completed.

### DXR-Hand Bone Measure

Digital X-ray radiogrammetry (DXR) (Sectra, Linkoping, Sweden) was used to measure hand bone mineral density (BMD) and metacarpal cortical index (MCI) on the same digitized hand X-rays used for assessment of radiographic joint damage. DXR is a computer version of the traditional radiogrammetry technique,[13] and have improved the method and its precision substantially. DXR has been described in detail.[14,15,16] On hand radiographs, the computer automatically recognizes regions of interest (ROI) around the narrowest part of the second, third, and fourth metacarpal bone and measures cortical thickness, bone width, and porosity 118 times per cm. DXR-BMD is defined as: c X VPAcomb X (1-p), where c is a constant (determined by the result that DXR-BMD, on average, is equal to the mid-distal forearm region of the Hologic QDR-2000 device); VPA is volume per area; and p is porosity. DXR-MCI is defined as the combined cortical thickness divided by the outer cortical diameter and is a relative bone measure independent of bone size, bone length, and image capture setting.[16.17] Both DXR-BMD and DXR-MCI provide substantial degrees of precision.[17]

DXR-BMD was intended to be the main outcome measure in this study. However, many radiographs could not be analysed for BMD because of unknown image resolution. Generally, the equation for DXR-BMD is based on volume per area and requires a defined or known resolution, since a distance in a digitised radiograph cannot be measured when the resolution is unknown. Thus, DXR-MCI, which is a relative measure independent of image resolution, was used as the primary outcome measure. The correlation between DXR-BMD and DXR-MCI has been shown to be substantial (r>0.90), both cross-sectionally [18] and longitudinally. [19]

For comparison, results for DXR-BMD are also provided. All images of unknown resolution were analyzed by assuming 254 dpi (the scanning resolution for the radiographs before scoring). Several of the radiographs were, however, clearly of a resolution other than 254 dpi, most likely because they had been printed in non-true size before scanning. All available images from baseline were analyzed, as well as 26, 52 and 104 weeks, by DXR-BMD and calculated mean metacarpal width. Based on analyses from other studies with a controlled resolution, [19] a deviation from baseline width greater than 2% was believed to likely indicate an incorrect value. With this 2% value as a cut-off, 23% of the radiographs were excluded from further DXR-BMD analyses. The flow chart in FIG. 1 illustrates the patients who were included in the DXR-MCI and DXR-BMD analyses.

To avoid bias regarding dominant vs. non-dominant hand and to achieve better accuracy, mean value measurements from both hands were used.[10] If the radiograph from one hand could not be analysed or were missing, the radiograph from the available hand for all analyses at the different time points was used.

### Statistical Analysis

Since the data were not normally distributed, non-parametric analyses were conducted. No imputations were performed. Baseline values were compared between treatment groups with the Kruskall-Wallis method for continuous variables and with the chi-squared method for categorical variables. Comparisons of changes in hand BMD were conducted following methodologies parallel and similar to ones employed in the original Study J. [6] Two groups were compared in a hierarchical order with the Mann-Whitney U test (i.e., two-sided comparison of the combination group vs. MTX, followed by two-sided comparisons between the monotherapy treatment arms, and finally two-sided comparisons between the adalimumab monotherapy and the combination group). Each pair-wise comparison was completed only if the previous comparison was statistically significant. Bone loss over time was expressed as a negative value.

A linear regression model was developed to search for predictors of hand BMD loss at 104 weeks. Spearman correlation analyses were conducted in an attempt to correlate changes in DXR-MCI at 104 weeks with the following baseline variables: disease duration; disease activity measured by DAS28; [20] CRP; disability index of Health Assessment Questionnaire (HAQ DI) scores; [21] previous use of DMARDs and cortisone; radiographic joint damage; randomised treatment arm; and absolute DXR-MCI value. The variables with a p-value less than 0.15 were included in the multivariate model, which also was adjusted for age and sex. Treatment arm was coded as a dummy variable (MTX as 0, adalimumab as 1 and combination group as 2).

### Study Oversight and Ethics

As reported, Study J was approved by a central institutional review board and independent ethics committee at each participating site.[6]

### Results

Baseline DXR-MCI values were available for 768 of the 799 patients enrolled in Study J, and DXR-MCI values were missing for 2 of 539 patients who completed the study (FIG. 1). The corresponding numbers for available DXR-BMD data (based on the cut-off values for image resolution described in “Methods”) were 765 and 369, respectively (FIG. 1). Demographics and baseline clinical characteristics were comparable between the three treatment groups (Table 1).

TABLE 1
Baseline characteristics for early rheumatoid arthritis patients in Study J*

| | Adalimumab + | | Adalimumab | | Methotrexate | |
|---|---|---|---|---|---|---|
| | Methotrexate | | Monotherapy | | Monotherapy | |
| | (N = 261) | | (N = 261) | | (N = 246) | |
| Demographic characteristics | | | | | | |
| Age, years | 52.2 | (13.8) | 51.9 | (13.7) | 51.9 | (13.3) |
| Female, no. (%) | 187 | (71.6) | 205 | (78.5) | 181 | (73.6) |
| Clinical characteristics | | | | | | |
| Disease duration, years | 0.7 | (0.8) | 0.7 | (0.8) | 0.8 | (0.9) |
| Previously taken DMARDs, | 84 | (32.2) | 87 | (33.3) | 78 | (31.7) |
| no. (%) | | | | | | |
| Previously taken | 92 | (35.2) | 94 | (36.0) | 85 | (34.6) |
| corticosteroids, | | | | | | |
| no. (%) | | | | | | |
| Tender joint count, 0-66 | 31.1 | (14.1) | 31.7 | (13.5) | 32.2 | (14.3) |
| Swollen joint count, 0-66 | 21.2 | (11.1) | 21.7 | (10.2) | 21.6 | (11.3) |
| C-reactive protein, mg/l | 39.5 | (42.4) | 40.7 | (38.6) | 40.6 | (41.2) |
| HAQ, 0-3 | 1.5 | (0.6) | 1.6 | (0.6)** | 1.5 | (0.7) |
| DAS28 | 6.3 | (0.9) | 6.4 | (0.9) | 6.3 | (0.9) |
| Image analysis | | | | | | |
| Modified TSS | | | | | | |
| Mean | 18.1 | (20.3) | 18.4 | (18.2) | 21.5 | (21.8) |
| Median (25-75 percentile) | 12.8 | (6.0-24.0) | 13.5 | (5.1-25.5) | 15.5 | (7.5-28.5) |
| DXR-MCI | 0.45 | (0.09) | 0.45 | (0.09) | 0.46 | (0.08) |
| DXR-BMD, g/cm² | 0.57 | (0.08) | 0.57 | (0.08) | 0.58 | (0.08) |

*Except where indicated results are given in mean ± standard deviation for continuous variables and numbers and percentages for categorical variables.
**Significantly greater values in the adalimumab group compared to both the methotrexate and the combination group.
RA = rheumatoid arthritis; DMARDs = disease-modifying antirheumatic drugs; HAQ = Health Assessment Questionnaire; DAS28 = 28-joint disease activity score; TSS = total Sharp score; DXR = digital X-ray radiogrammetry; BMD = bone mineral density; MCI = metacarpal cortical index.

The only statistically significant difference between treatment arms was a slightly greater mean HAQ score for the adalimumab monotherapy group. Prior to enrollment, corticosteroids had been used by 35% of the patients (mean daily dosage of prednisolone was 6.6 mg), and 32% had been treated with traditional DMARDs other than MTX. The baseline radiographic damage scores were similar across treatment groups, with a median (mean) Sharp score of 14.0 (19.3) (Table 1).

Median percentage DXR-MCI changes for all patients were −1.29, −2.45 and −3.72 after 26, 52 and 104 weeks, respectively. Corresponding values for DXR-BMD were −1.07%,−1.72% and −2.63%. These changes from baseline in DXR-MCI and DXR-BMD were significant for all subgroups at all time points during follow-up (p<0.001 for all). Use of corticosteroids or DMARDs did not affect hand bone loss (data not shown).

Correlations coefficients (r) between the DXR-MCI and the DXR-BMD changes were 0.88, 0.93 and 0.94 at 26, 52 and 104 weeks, respectively (p<0.001 for all).

### DXR-MCI Changes Between Treatment Arms

At 26, 52, and 104 weeks median percentage DXR-MCI changes were −1.15, −2.16, and −3.03 for the adalimumab-plus-MTX combination group; −1.33, −2.45, and −4.03 for the adalimumab monotherapy group; and −1.42, −2.87, and −4.62 for the MTX monotherapy group (FIG. 2).

The rate of DXR-MCI loss was significantly greater for the MTX group compared with the combination group at 52 weeks (p=0.009) and 104 weeks (p<0.001), and the same trend was also observed at 26 weeks (p=0.19). Bone loss in the adalimumab group was also numerically lower than in the MTX group at 104 weeks (p=0.10).

### DXR-BMD Changes Between Treatment Arms

The median DXR-BMD percentage changes in the combination group were −1.06 at 26 weeks, −1.63 at 52 weeks, and −2.49 at 104 weeks. In the adalimumab group, the respective changes at 26, 52, and 104 weeks were −0.96, −1.97, and −2.40; and for the MTX group, the changes were −1.20, −1.86, and −3.58. A significant difference between the DXR-BMD change in the MTX group and the combination group at 104 weeks (p=0.049) were observed and a trend towards a significance difference at 52 weeks (p=0.10). In addition, a trend towards a difference between the MTX and adalimumab groups was observed for 104-week values (p=0.16).

### DXR-MCI and Radiographic Damage

The median (mean) radiographic changes in the modified Sharp score at 26, 52, and 104 weeks respectively were 0 (0.5), 0 (0.9), and 0 (1.0) for the combination group; and 0.5 (2.1), 0.5 (3.3), and 1.0 (4.8) for the adalimumab monotherapy group. For the MTX monotherapy group, the respective changes were 1.0 (3.4), 2.0 (5.1), and 2.0 (6.4) (FIG. 2). The discrepancy in the results of this analysis vs. findings of the original Study J is likely a result of the slight differences in number of study participants (FIG. 1), and the fact that no imputations were conducted here. The correlations (r) between DXR-MCI change and change in Sharp score at 26, 52, and 104 weeks were r=−0.12 (p=0.001); r=−0.23 (p<0.001); and r=−0.32 (p<0.001). Comparable r-values for correlations between DXR-BMD and Sharp score changes were −0.15, −0.23, and −0.33, respectively (p<0.001 for all).

### Multivariate Model

The variables included in the final multivariate model were baseline values of disease duration, DAS28 score, CRP, DXR-MCI, HAQ, radiographic damage, and treatment group (dummy variable), together with age and sex.

Older age, greater CRP and non-use of adalimumab turned out to be independent predictors for cortical hand bone loss, while greater DAS28 scores (i.e., greater disease severity) (p=0.07), and shorter disease durations (p=0.11) were trending towards statistical significance within model (Table 2).

TABLE 2
Predictors for percentage DXR-MCI loss at 104-weeks
of follow-up for 515 RA arthritis patients explored
by multivariate linear regression model

| | DXR-MCI percentage changes at 104 weeks | |
|---|---|---|
| | Beta | p-value |
| Age, years | −0.25 | <0.001 |
| Female | −0.04 | 0.36 |
| Disease duration, years | 0.06 | 0.11 |
| C-reactive protein, mg/l | −0.23 | <0.001 |
| DAS28 | −0.09 | 0.07 |
| Treatment group* | 0.16 | <0.001 |

| | |
|---|---|
| R², adjusted | 0.19 |

*Treatment group coded as a dummy variable: 0 = MTX, 1 = adalimumab, 2 = adalimumab plus MTX. MCI baseline, Sharp score baseline, and HAQ did not influence the model.
RA = rheumatoid arthritis; DXR = digital X-ray radiogrammetry; MCI = metacarpal cortical index; HAQ = Health Assessment Questionnaire; DAS28 = 28-joint Disease Activity Score.;

The key finding of this analysis was that anti-TNF therapy with adalimumab in combination with MTX provided better bone protection than either adalimumab or MTX monotherapies in patients with a certain type of RA, i.e., early, aggressive RA. The order of hand bone loss across the three treatment arms was the same as has been observed for overall radiographic damage in Study J (FIG. 2). Further, the results from the multivariate model highlighted the importance of inflammation (assessed with CRP) as the driving force for bone damage in active RA and the importance of TNF involvement in this process.

With respect to mechanism, the present analysis supports the hypothesis that both erosions and osteoporosis are a result of the same pathophysiological mechanism, which includes activation of the osteoclast cell. This hypothesis is based on findings from both animal [2,22] and human studies.[3] The osteoclasts that are the main cells for bone degradation are driven by the synovial inflammation and stimulated by TNF, macrophage colony-stimulating factor (M-CSF), and receptor activator of nuclear factor-K ligand (RANKL). These cytokines activate the osteoclast that then causes osteoporosis (localised and generalised) and erosions.[23]

The findings from this study support the fact that suppressing inflammation through anti-TNF therapy reduces hand bone loss. Moreover, from the multivariate model that was conducted, CRP proved to be a strong predictor for loss of DXR-MCI.

While not wishing to be bound by theory, bone loss in the combination group (adalimumab and MTX) may be attributable, at least in part, to the substantial disease activity in the early RA patients participating in Study J, and their poor prognosis in terms of bone damage (rheumatoid factor-positivity and erosive disease). [24]

While the positive effects of TNF-antagonist therapy in active RA seemed to have been more pronounced for radiographic joint damage than for hand bone mass (FIG. 2), TNF-antagonist therapy was still found to reduce the risk of developing erosions and the rate of inflammatory-related hand bone loss. While not wishing to be bound by theory, one explanation for this discrepancy may be that conventional radiographs are not sensitive enough to detect bone damage. Both ultrasound (US) and magnetic resonance imaging (MRI) have been demonstrated to be more sensitive than radiographs in detecting erosions.25] Further, MRI can detect erosions years before they become visible on radiographs. [26] In addition, MRI synovitis has even been detected in RA patients in the state of both clinical and radiographic remission (“true remission”).[27] Hand bone loss assessed by DXA has also been shown to be a more sensitive marker for bone damage than conventional radiographs.[10] Therefore, the combination of ever-present inflammation in patients with greater disease activity, as well as the ability of DXR to detect small changes in bone mass, may explain ongoing loss of hand bone, even in the combination therapy group. It's also important to note the influence of normal bone loss that takes place also in healthy adults, especially postmenopausal women. Normal bone loss for DXR-MCI has been examined only in cross-sectional studies reporting an annual rate of bone loss between 0.7-0.9%.[16,28,29]

When this analysis was planned, radiographs primarily for DXR-BMD were originally to be analyzed. However, for reasons described in “Methods,” there were difficulties in analysing a notable percentage of the radiographs for DXR-BMD. This study was based on post-hoc analyses of Study J. By using the relative DXR-MCI measure instead of the absolute measure of BMD, the opportunity to correct for porosity was unavailable. Further, DXR-BMD, as opposed to DXR-MCI, is calibrated for blurring and particular qualities of the different radiographic measurement equipment. However, DXR has improved the precision of MCI, [17] and there is a strong correlation between DXR-BMD and DXR-MCI (r>0.9).[18,19] DXR-MCI and DXR-BMD have also been found to be greatly correlated with DXA-BMD.[19] These facts suggest that DXR-MCI is a valid surrogate for measuring hand bone mass change.

There was little information available on the use of bisphosphonates in patients participating in Study J, however, the study design of a double-blind, randomised controlled trial minimised the effect of potential bias. In addition, zoledronic acid was not on the market for osteoporosis treatment when Study J was conducted. Further, in another study, the positive effect of infliximab in suppressing inflammation on bone was found to be independent of bisphosphonates. [7]

In conclusion, this study provides evidence that potent anti-TNF therapy not only reduces the risk of developing erosions, but also reduces the rate of inflammatory-related hand bone loss in RA. This study also suggests that the bone damage disease process may still present in RA patients treated with TNF antagonists, even if joint damage observed on radiographs appears to be arrested.

### B. Adalimumab Reduces Hand Bone Loss in Rheumatoid Arthritis (RA) Independent of Clinical Response: Subanalysis of Study J

Adalimumab reduces the rates of both radiographic joint damage and hand bone loss in patients with early RA. The rate of radiographic joint progression has been shown to be reduced independent of patient's clinical response to adalimumab. This has previously not been examined for hand bone loss, the second feature of bone involvement in inflammatory RA.

The objective of the study described herein was to examine the relationship between hand bone loss and clinical response in patients receiving methotrexate (MTX) monotherapy and in patients receiving adalimumab plus MTX in Study J.

### Methods

As described above, Study J compared the efficacy of adalimumab plus MTX vs. MTX alone and adalimumab alone in early (<3 yrs), active, MTX-naive RA patients. The subanalysis described herein involved the MTX monotherapy and the combination therapy groups. Hand bone loss was assessed by digital X-ray radiogrammetry metacarpal cortical index (DXR-MCI), calculated from digitized radiographs (DXR, Sectra, Sweden). MCI, defined as the combined metacarpal cortical thickness divided by the outer bony diameter, has been shown to be well-correlated with bone mineral density. MCI percentage change from baseline to 52 weeks was evaluated for patients with different clinical response. Disease activity was assessed by DAS28 scores at 52 weeks in 4 subgroups: Remission=DAS28<2.60; Low disease activity=DAS28 2.61-3.20; Moderate disease activity=DAS28 3.21-5.20; and high disease activity=DAS28>5.20. Non-parametric group comparisons were performed.

### Results

For the combination therapy group (MTX and adalimumab), there was no difference in bone loss among RA patients with remission, low, moderate, and high disease activity (p=0.97). For the MTX group, there were numerical differences between the 4 clinical disease activity subgroups (p=0.10) (Table 3). Because of the small numbers of patients in some of the 4 subgroups (see Table 3), we further divided patients into 2 other subgroups: remission and low disease activity vs. moderate and high disease activity. In the MTX group, patients with moderate and high DAS28 lost significantly more DXR-MCI than patients with low DAS 28 (−4.65 vs.−2.99, p=0.01), while no statistically significant difference was seen in the combination therapy group (−3.10 vs.−2.70, p=0.99). Correlation between disease activity and hand bone loss (percentage DXR-MCI) was −0.14 (p=0.06) in the MTX group and −0.07 (p=0.33) for the combination therapy group.

TABLE 3
Differences in bone loss among RA patients with remission,
low, moderate, and high disease activity treated with MTX
alone, or with Adalimumab + MTX (combination therapy)

| | MTX | | Adalimumab + MTX | |
|---|---|---|---|---|
| | | Mean (median) | | Mean (median) |
| | | % DXR-MCI | | % DXR-MCI |
| | N = 185 | change | N = 208 | change |
| Remission | 51 | −2.99 (−2.14) | 110 | −2.72 (−2.44) |
| Low disease | 31 | −2.98 (−2.20) | 38 | −2.62 (−1.94) |
| activity | | | | |
| Moderate disease | 79 | −4.65 (−3.33) | 51 | −3.17 (2.01) |
| activity | | | | |
| High disease | 24 | −4.64 (−3.02) | 9 | −2.72 (−1.63) |
| activity | | | | |

In conclusion, these data suggest that adalimumab reduces hand bone loss independent of clinically assessed disease activity as previously shown for radiographic joint damage. These results support the hypothesis that TNF influences bone loss not only by stimulating RANKL by inflammation, but also by activating the osteoclast directly.

### REFERENCES

1. Arnett F C, Edworthy S M, Bloch D A, McShane D J, Fries J F, Cooper N S, et al. The American Rheumatism Association 1987 revised criteria for the classification of rheumatoid arthritis. Arthritis Rheum 1988; 31:315-24.
2. Pettit A R, Ji H, von Stechow D, Muller R, Goldring S R, Choi, Y et al. TRANCE/RANKL knockout mice are protected from bone erosion in a serum transfer model of arthritis. Am J Pathol 2001; 159:1689-99.
3. Jarrett S J, Conaghan P G, Sloan V S, Papanastasiou P, Ortmann C E, O'Connor P J, et al. Preliminary evidence for a structural benefit of the new bisphosphonate zoledronic acid in early rheumatoid arthritis. Arthritis Rheum 2006; 54:1410-14.
4. St Clair E W, van der Heijde D M, Smolen J S, Maini R N, Bathon J M, Emery P, et al. Combination of infliximab and methotrexate therapy for early rheumatoid arthritis: a randomized, controlled trial. Arthritis Rheum 2004; 50:3432-43.
5. van der Heijde D M, Klareskog L, Rodriguez-Valverde V, Codreanu C, Bolosiu H,
Melo-Gomes J, et al. Comparison of etanercept and methotrexate, alone and combined, in the treatment of rheumatoid arthritis: two-year clinical and radiographic results from the TEMPO study, a double-blind, randomized trial. Arthritis Rheum 2006; 54:1063-74.
6. Breedveld F C, Weisman M H, Kavanaugh A F, Cohen S B, Pavelka K, van Vollenhoven R, et al. The PREMIER study: A multicenter, randomized, double-blind clinical trial of combination therapy with adalimumab plus methotrexate versus methotrexate alone or adalimumab alone in patients with early, aggressive rheumatoid arthritis who had not had previous methotrexate treatment. Arthritis Rheum 2006; 54:26-37.
7. Marotte H, Pallot-Prades B, Grange L, Gaudin P, Alexandre C, Miossec P. A 1-year case-control study in patients with rheumatoid arthritis indicates prevention of loss of bone mineral density in both responders and nonresponders to infliximab. Arthritis Res Ther 2007; 9:R61.
8. Lange U, Teichmann J, Muller-Ladner U, Strunk J. Increase in bone mineral density of patients with rheumatoid arthritis treated with anti-TNF-alpha antibody: a prospective open-label pilot study. Rheumatology 2005; 44:1546-8.
9. Vis M, Havaardsholm E A, Haugeberg G, Uhlig T, Voskuyl A E, van der Stadt R J, et al. Evaluation of bone mineral density, bone metabolism, osteoprotegerin and receptor activator of the NFkappaB ligand serum levels during treatment with infliximab in patients with rheumatoid arthritis. Ann Rheum Dis 2006; 65:1495-9.
10. Haugeberg G, Green M J, Conaghan P G, Quinn M, Wakefield R, Proudman S M, et al. Hand bone densitometry: a more sensitive standard for the assessment of early bone damage in rheumatoid arthritis. Ann Rheum Dis 2007; 66:1513-17.
11. Seriolo B, Paolino S, Sulli A, Ferretti V, Cutolo M. Bone metabolism changes during anti-TNF-alpha therapy in patients with active rheumatoid arthritis. Ann NY Acad Sci 2006; 1069:420-7.
12. Haugeberg G, Strand A, Kvien T K, Kirwan J R. Reduced loss of hand bone density with prednisolone in early rheumatoid arthritis: results from a randomized placebo-controlled trial. Arch Intern Med 2005; 165:1293-7.
13. Barnett E, Nordin B E. The radiological diagnosis of osteoporosis: a new approach. Clin Radiol 1960; 11:166-74.
14. Rosholm A, Hyldstrup L, Backsgaard L, Grunkin M, Thodberg H H. Estimation of bone mineral density by digital X-ray radiogrammetry: theoretical background and clinical testing. Osteoporos Int 2001; 12:961-9.
15. Jorgensen J T, Andersen P B, Rosholm A, Bjamason N H. Digital X-ray radiogrammetry: a new appendicular bone densitometric method with high precision. Clin Physiol 2000; 20:330-5.
16. Hyldstrup L, Nielsen S P. Metacarpal index by digital X-ray radiogrammetry: normative reference values and comparison with dual X-ray absorptiometry. Clin Densitom 2001; 4:299-306.
17. Nielsen S P. The metacarpal index revisited: a brief overview. J Clin Densitom 2001; 4:199-207.
18. Bottcher J, Malich A, Pfeil A, Petrovitch A, Lehmann G, Heyne J P, et al. Potential clinical relevance of digital radiogrammetry for quantification of periarticular bone demineralization in patients suffering from rheumatoid arthritis depending on severity and compared with DXA. Eur Radiol 2004; 14:631-7.
19. Hoff M, Haugeberg G, Kvien T K. Hand bone loss as an outcome measure in established rheumatoid arthritis: 2-year observational study comparing cortical and total bone loss. Arthritis Res Ther 2007; 9:R81.
20. Prevoo M L, van't Hof M A, Kuper H H, van Leeuwen M A, van de Putte L B, van Riel P L. Modified disease activity scores that include twenty-eight-joint counts. Development and validation in a prospective longitudinal study of patients with rheumatoid arthritis. Arthritis Rheum 1995; 38:44-8.
21. Fries J F, Spitz P, Kraines R G, Holman H R. Measurement of patient outcome in arthritis. Arthritis Rheum 1980; 23:137-45.
22. Redlich K, Hayer S, Ricci R, David J P, Tohidast-Akrad M, Kollias G, et al. Osteoclasts are essential for TNF-alpha-mediated joint destruction. J Clin Invest 2002; 110:1419-27.
23. Romas E, Gillespie M T. Inflammation-induced bone loss: can it be prevented? Rheum Dis Clin North Am 2006; 32:759-73.
24. Emery P. The Dunlop-Dottridge Lecture: prognosis in inflammatory arthritis: the value of HLA genotyping and the oncological analogy. J Rheumatol 1997; 24:1436-42.
25. Dohn U M, Ejbjerg B J, Court-Payen, Hasselquist M, Narvestad E, Szkularek M, et al. Are bone erosions detected by magnetic resonance imaging and ultrasonography true erosions? A comparison with computed tomography in rheumatoid arthritis metacarpophalangeal joints. Arthritis Res Ther 2006; 8:R110.
26. Ostergaard M, Hansen M, Stoltenberg M, Jensen K E, Szkudlarek M, Pedersen-Zbinden B, et al. New radiographic bone erosions in the wrists of patients with rheumatoid arthritis are detectable with magnetic resonance imaging a median of two years earlier. Arthritis Rheum 2003; 48:2128-31.
27. Brown A K, Quinn M A, Karim Z, Conaghan P G, Peterfy C G, Hensor E, et al. Presence of significant synovitis in rheumatoid arthritis patients with disease-modifying antirheumatic drug-induced clinical remission: evidence from an imaging study may explain structural progression. Arthritis Rheum 2006; 54:3761-73.
28. Bottcher J, Pfeil A, Schafer M L, Petrovitch A, Seidl B E, Mentzel H J, et al. Normative data for digital X-ray radiogrammetry from a female and male German cohort. J Clin Densitom 2006; 9:341-50.
29. Toledo V A, Jergas M. Age-related changes in cortical bone mass: data from a German female cohort. Eur Radiol 2006; 16:811-17.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents, and published patent applications, and patent applications cited throughout this application are incorporated herein by reference.

What is claimed:

1. A method for treating bone loss in a subject, comprising administering a human TNFα antibody, or antigen-binding portion thereof, to the subject such that bone loss is treated.

2. The method of claim 1, wherein the subject has rheumatoid arthritis.

3. The method of claim 2, wherein the treatment further comprises administration of methotrexate.

4. The method of claim 1, wherein hand bone loss is treated.

5. The method of claim 1, wherein the human TNFα antibody, or an antigen-binding portion thereof, is selected from the group consisting of
a) a human antibody, or antigen-binding portion thereof, that dissociates from human TNFα with a Kd of 1×10⁻⁸ M or less and a Koff rate constant of 1×10⁻³ s⁻¹ or less, both determined by surface plasmon resonance, and neutralizes human TNFα cytotoxicity in a standard in vitro L929 assay with an IC50 of 1×10⁻⁷ M or less;
b) a human antibody, or antigen-binding portion thereof, having the following characteristics:
i) dissociates from human TNFα with a Koff rate constant of 1×10⁻³ s⁻¹ or less, as determined by surface plasmon resonance;
ii) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 8 and/or 9;
iii) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12,
c) a human TNFα antibody, or antigen-binding portion thereof, comprising a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprising a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11;
d) a human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2;
e) adalimumab; and
f) golimumab.

6. The method of claim 1, wherein the subject was previously selected as having or at risk of having bone loss.

7. A method for treating hand bone loss in a subject, comprising administering a TNFα inhibitor to the subject, such that hand bone loss is treated.

8. The method of claim 7, wherein the subject has rheumatoid arthritis.

9. The method of claim 8, wherein the treatment further comprises administration of methotrexate.

10. The method of claim 7, wherein the subject has osteoporosis.

11. The method of claim 7, wherein the subject has osteoarthritis.

12. The method of claim 7, wherein cortical hand bone loss is treated.

13. The method of claim 7, wherein the TNFα inhibitor is a TNFα antibody, or antigen-binding portion thereof.

14. The method of claim 13, wherein the TNFα antibody, or antigen-binding portion thereof, is a human TNFα antibody, or antigen-binding portion thereof.

15. The method of claim 14, wherein the human TNFα antibody, or an antigen-binding portion thereof, is selected from the group consisting of
a) a human antibody, or antigen-binding portion thereof, that dissociates from human TNFα with a Kd of 1×10-8 M or less and a Koff rate constant of 1×10-3 s-1 or less, both determined by surface plasmon resonance, and neutralizes human TNFα cytotoxicity in a standard in vitro L929 assay with an IC50 1×10-7 M or less;
b) a human antibody, or antigen-binding portion thereof, having the following characteristics:
i) dissociates from human TNFα with a Koff rate constant of 1×10-3 s⁻¹ or less, as determined by surface plasmon resonance;
ii) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 8 and/or 9;
iii) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12,
c) a human TNFα antibody, or antigen-binding portion thereof, comprising a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprising a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11;
d) a human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2;
e) adalimumab; and
f) golimumab.

16. The method of claim 7, wherein the subject was previously selected as having or at risk of having bone loss.

17. A method for treating hand bone loss in a subject, comprising selecting a subject who has hand bone loss or is at risk of having hand bone loss, and administering a TNFα inhibitor to the subject, such that hand bone loss is treated.

18. The method of claim 17, wherein the subject has rheumatoid arthritis.

19. The method of claim 18, wherein the treatment further comprises administration of methotrexate.

20. The method of claim 17, wherein the subject has osteoporosis.

21. The method of claim 17, wherein the subject has osteoarthritis.

22. The method of claim 17, wherein cortical hand bone loss is treated.

23. The method of claim 17, wherein the TNFα inhibitor is a TNFα antibody, or antigen-binding portion thereof.

24. The method of claim 23, wherein the TNFα antibody, or antigen-binding portion thereof, is a human TNFα antibody, or antigen-binding portion thereof.

25. The method of claim 24, wherein the human TNFα antibody, or an antigen-binding portion thereof, is selected from the group consisting of
a) a human antibody, or antigen-binding portion thereof, that dissociates from human TNFα with a Kd of 1×10-8 M or less and a Koff rate constant of 1×10-3 s-1 or less, both determined by surface plasmon resonance, and neutralizes human TNFα cytotoxicity in a standard in vitro L929 assay with an IC50 of 1×10-7 M or less;
b) a human antibody, or antigen-binding portion thereof, having the following characteristics:
i) dissociates from human TNFα with a Koff rate constant of 1×10-3 s-1 or less, as determined by surface plasmon resonance;
ii) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 8 and/or 9;
iii) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12,
c) a human TNFα antibody, or antigen-binding portion thereof, comprising a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprising a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11;
d) a human TNFα antibody, or antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2;
e) adalimumab; and
f) golimumab.


SEQUENCE LISTING (37 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  adalimumab light chain variable region CDR3
QRYNRAPYX

SEQ ID NO: 4 - protein, 12 aa, Artificial Sequence
  adalimumab heavy chain variable region CDR3
VSYLSTASSLDX

SEQ ID NO: 5 - protein, 7 aa, Artificial Sequence
  adalimumab light chain variable region CDR2
AASTLQS

SEQ ID NO: 6 - protein, 17 aa, Artificial Sequence
  adalimumab heavy chain variable region CDR2
AITWNSGHIDYADSVEG

SEQ ID NO: 7 - protein, 11 aa, Artificial Sequence
  adalimumab light chain variable region CDR1
RASQGIRNYLA

SEQ ID NO: 8 - protein, 5 aa, Artificial Sequence
  adalimumab heavy chain variable region CDR1
DYAMH

SEQ ID NO: 9 - protein, 107 aa, Artificial Sequence
  2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  2SD4 light chain variable region CDR3
QKYNSAPYA

SEQ ID NO: 12 - protein, 9 aa, Artificial Sequence
  EP B12  light chain variable region CDR3
QKYNRAPYA

SEQ ID NO: 13 - protein, 9 aa, Artificial Sequence
  VL10E4 light chain variable region CDR3
QKYQRAPYT

SEQ ID NO: 14 - protein, 9 aa, Artificial Sequence
  VL100A9 light chain variable region CDR3
QKYSSAPYT

SEQ ID NO: 15 - protein, 9 aa, Artificial Sequence
  VLL100D2 light chain variable region CDR3
QKYNSAPYT

SEQ ID NO: 16 - protein, 9 aa, Artificial Sequence
  VLL0F4 light chain variable region CDR3
QKYNRAPYT

SEQ ID NO: 17 - protein, 9 aa, Artificial Sequence
  LOE5 light chain variable region CDR3
QKYNSAPYY

SEQ ID NO: 18 - protein, 9 aa, Artificial Sequence
  VLLOG7 light chain variable region CDR3
QKYNSAPYN

SEQ ID NO: 19 - protein, 9 aa, Artificial Sequence
  VLLOG9 light chain variable region CDR3
QKYTSAPYT

SEQ ID NO: 20 - protein, 9 aa, Artificial Sequence
  VLLOH1 light chain variable region CDR3
QKYNRAPYN

SEQ ID NO: 21 - protein, 9 aa, Artificial Sequence
  VLLOH10 light chain variable region CDR3
QKYNSAAYS

SEQ ID NO: 22 - protein, 9 aa, Artificial Sequence
  VL1B7 light chain variable region CDR3
QQYNSAPDT

SEQ ID NO: 23 - protein, 9 aa, Artificial Sequence
  VL1C1 light chain variable region CDR3
QKYNSDPYT

SEQ ID NO: 24 - protein, 9 aa, Artificial Sequence
  VL0.1F4 light chain variable region CDR3
QKYISAPYT

SEQ ID NO: 25 - protein, 9 aa, Artificial Sequence
  VL0.1H8 light chain variable region CDR3
QKYNRPPYT

SEQ ID NO: 26 - protein, 9 aa, Artificial Sequence
  LOE7.A light chain variable region CDR3
QRYNRAPYA

SEQ ID NO: 27 - protein, 12 aa, Artificial Sequence
  2SD4 heavy chain variable region CDR3
ASYLSTSSSLDN

SEQ ID NO: 28 - protein, 12 aa, Artificial Sequence
  VH1B11 heavy chain variable region CDR3
ASYLSTSSSLDK

SEQ ID NO: 29 - protein, 12 aa, Artificial Sequence
  VH1D8 heavy chain variable region CDR3
ASYLSTSSSLDY

SEQ ID NO: 30 - protein, 12 aa, Artificial Sequence
  VH1A11 heavy chain variable region CDR3
ASYLSTSSSLDD

SEQ ID NO: 31 - protein, 12 aa, Artificial Sequence
  VH1B12 heavy chain variable region CDR3
ASYLSTSFSLDY

SEQ ID NO: 32 - protein, 12 aa, Artificial Sequence
  VH1E4 heavy chain variable region CDR3
ASYLSTSSSLHY

SEQ ID NO: 33 - protein, 12 aa, Artificial Sequence
  VH1F6 heavy chain variable region CDR3
ASFLSTSSSLEY

SEQ ID NO: 34 - protein, 12 aa, Artificial Sequence
  3C-H2 heavy chain variable region CDR3
ASYLSTASSLEY

SEQ ID NO: 35 - protein, 12 aa, Artificial Sequence
  VH1-D2.N heavy chain variable region CDR3
VSYLSTASSLDN

SEQ ID NO: 36 - DNA, 321 bp, Artificial Sequence
  adalimumab light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  adalimumab heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt